(19) United States
(12) Patent Application Publication
(10) Pub. No.: US 2003/0143571 A1
(43) Pub. Date: Jul. 31, 2003

(54) Infectious disease microarray

(75) Inventors: Nicholas J.H. Sharp, Vancouver (CA); Scott J. Schatzberg, Ithica, NY (US); Gregory Robert OBrian, Raleigh, NC (US)

Correspondence Address: JENKINS & WILSON, PA, 3100 TOWER BLVD, SUITE 1400, DURHAM, NC, 27707, US

(73) Assignee: North Carolina State University, Raleigh, NC (US)

(21) Appl. No.: 10/215,314

(22) Filed: Aug. 8, 2002

Related U.S. Application Data
(60) Provisional application No. 60/310,985, filed on Aug. 8, 2001.

(51) Int. Cl. C12Q 1/68; C12Q 1/70

(52) U.S. Cl. 435/6; 435/5

(57) ABSTRACT

A method for detecting one or more pathogens in a subject. The method includes the steps of: (a) procuring a biological sample, wherein the biological sample comprises nucleic acid material; (b) amplifying the nucleic acid material using random primers to produce a set of random amplicons; (c) providing one or more pathogen-specific probes or probe sets; (d) hybridizing the set of random amplicons with the one or more pathogen-specific probes or probe sets; and (e) determining selective hybridization between a random amplicon and a pathogen-specific probe or probe set, whereby the presence of a pathogen in a biological sample is detected.

### CROSS REFERENCE TO RELATED APPLICATIONS

The present patent application is based on and claims priority to U.S. Provisional Application Serial No. 60/310,985, entitled “INFECTIOUS DISEASE MICROARRAY”, which was filed Aug. 8, 2001 and is incorporated herein by reference.

### FIELD OF THE INVENTION

The present invention generally relates to detection of a pathogen in a biological sample. More particularly, the present invention provides a method for hybridizing a collection of random amplicons derived from a nucleic acid sample with one or more pathogen-specific probes.

### TABLE OF ABBREVIATIONS

DOP-PCR—degenerate oligonucleotide primed polymerase chain reaction

EST—expressed sequence tag

HRP—horse radish peroxidase

PCR—polymerase chain reaction

PEP—primer extension polymerase chain reaction

PNA—peptide nucleic acid

RAPD—rapid amplification of polymorphic DNA

RP-PCR—random-primed polymerase chain reaction

SISPA—sequence-independent, single primer amplification

STS—sequence tagged site

TSA™—Tyramide Signal Amplification

### BACKGROUND ART

The last two decades have seen the nearly monthly discovery of new infectious agents or evolution of existing agents that have challenged health care, agricultural, and environmental protection workers worldwide. See Morse (1995) Emerg Infect Dis 1:7-15; Lederberg (1997) Emerg Infect Disease 4:417-423; and Morris & Potter (1997) Emerg Infect Dis 1:435-441. The ensuing resurgence of infections and diversity of infectious agents highlight the necessity for improved methods for prevention, diagnosis, and management of such conditions.

In the field of infectious disease diagnostics, PCR assays can rapidly and accurately detect the presence of microorganisms directly from clinical specimens, and commercial PCR assays for the diagnosis of some infectious agents are now routinely used in many diagnostic laboratories. Nucleic acid amplification techniques can also be used to determine viral load and thereby facilitate management of patients with a viral infection, for example HIV infection or hepatitis C infection. See Sakallah (2000) Biotechnol Annu Rev 6:141-161; Louie et al. (2000) CMAJ 163(3):301-309; Gasser (1999) Vet Parasitol 84(3-4):229-258; Dumler & Valsamakis (1999) Am J Clin Pathol 112 (1Suppl1):S33-39; and Specter et al., eds. (1998) Rapid Detection of Infectious Agents, Plenum Press, New York, N.Y.

DNA-based assays have partly replaced classic diagnostic methods that lacked sensitivity, specificity, or rapid analysis. Early outcome-based studies also suggest that molecular methods can provide substantial reductions in per patient costs when compared to epidemiological or other types of assessment. See Dumler & Valsmakis (1999) Am J Clin Pathol 112(1Suppl1):S33-39. Multiplex PCR-based assays have been developed and used for detection of multiple pathogens in a single PCR reaction. According to this technique, several pairs of gene-specific primers are used simultaneously. See e.g., Gröndahl et al. (1999) J Clin Microbiol 37:1-7; Ley et al. (1998) Eur J Clin Microbial Infect Dis 17:247-253; Goldenberger et al. (1997) J Clin Microbiol 35:2733-2739; and Klausegger et al. (1999) J Clin Microbiol 37:464-466.

Despite these advances, the detection of pathogens in clinical samples is often limited by the minute quantities of pathogen. See e.g., Louie et al. (2000) CMAJ 163(3):301-309. Further, current tests rely on an initial diagnosis that suspects the presence of a particular pathogen, and thus such tests are inappropriate for cases wherein an epidemiological diagnosis is unclear. See Snijders et al. (2000) J Clin Pathol 53:289-294; Elfath et al. (2000) Clin Microbiol Rev 13(4):559-570. In addition, the specificity of the test can be easily compromised by contamination of the specimen during laboratory processing. Contamination or amplification product carryover of even minute amounts of nucleic acid can be efficiently amplified using gene-specific primers and can lead to a false-positive test result. Methods for detecting a pathogen in other biological samples (e.g., plant samples, food samples, or environmental samples) are met with similar challenges.

Thus, there exists a long-felt need in the art for a method for detecting pathogens with improved sensitivity and versatility. Ideally, such a method can identify any pathogen, whose presence is suspected or unsuspected, in any biological sample.

To meet this need, the present invention discloses a method that incorporates random amplification and sequence-specific hybridization techniques. In accordance with the present invention, the method can be used to detect a pathogen in a biological sample, for example a clinical sample, a plant or plant parts, a food sample, or an environmental sample (such as a sample suspected of containing a pathogen associated with biological warfare or bioterrorism). In particular, the method enables a simultaneous survey of multiple potential pathogens in a biological sample, such that prior pathological diagnosis or a preponderance of the presence of a pathogen is not essential. The disclosed methods facilitate early detection of a pathogen, thereby reducing associated sequellae and improving successful amelioration of the infectious agent.

### SUMMARY OF INVENTION

The present invention discloses a method for detecting a pathogen in a biological sample. The method comprises: (a) procuring a biological sample, wherein the biological sample comprises nucleic acid material; (b) amplifying the nucleic acid material using random primers to produce a set of random amplicons; (c) providing one or more pathogen-specific probes; (d) hybridizing the set of random amplicons with the one or more pathogen-specific probes; and (e) determining selective hybridization between a random amplicon and a pathogen-specific probe, whereby the presence of a pathogen in the biological sample is detected.

The disclosed method for detecting a pathogen in a biological sample can also employ one or more pathogen-specific probe sets, wherein each of the one or more pathogen-specific probe sets comprises two or more pathogen-specific probes, each of the two or more pathogen-specific probes of a pathogen-specific probe set comprising a different nucleotide sequence of a same pathogen. Preferably, each of the one or more pathogen-specific probe sets comprises at least about four or five pathogen-specific probes, or more preferably about 6 to 10 pathogen-specific probes.

Representative biological samples that can be used in accordance with the disclosed methods include but are not limited to a clinical sample, a plant sample, an environmental sample, or a food sample. Thus, a pathogen can be detected in a clinical sample, preferably a clinical sample derived from a warm-blooded vertebrate, and more preferably a clinical sample derived from a human. Such a clinical sample can comprise blood; plasma; urine; stool; sputum; a biopsy; a lesional or ulcerous swab; pus; a throat, nose, or nasopharyngeal swab; mucus; an endotracheal aspirate; bronchoalveolar lavage; a conjuctival or corneal swab; bone marrow; cerebrospinal fluid; skin; hair; nails; a cell culture; any other clinical sample; or combinations thereof.

The detection methods of the present invention can also be used to detect a pathogen in a biological sample derived from a plant or plant parts, including a seed, a leaf, a stem, a root, a flower, a fruit, a plant culture, or combinations thereof.

Further provided is a method for detecting a pathogen in a biological sample derived from the environment, such as a soil sample or a water sample, and including but not limited to a sample suspected of containing a pathogen associated with biological warfare or bioterrorism.

Thus, a biological sample that can be analyzed in accordance with the present invention can be derived from any biological source comprising nucleic acid material, including deoxyribonucleic acid material, ribonucleic acid material, or a combination thereof. The amplified sample can further comprise a detectable label such as a fluorophore or an epitope. The detectable label can be incorporated during amplification of the sample or added following amplification of the sample.

The disclosed methods for detecting a pathogen in a biological sample, employ one or more pathogen-specific probes or probe sets, wherein each of the one or more pathogen-specific probes comprises a nucleotide sequence derived from a bacterium, a fungus, a virus, a protozoan, or a parasite. Optionally, a pathogen-specific probe can comprise a nucleotide sequence of a pathogen gene, including but not limited to a nucleotide sequence encoding a pathogen polypeptide.

In one embodiment, the one or more pathogen-specific probes or probe sets further comprises a detectable label, preferably a fluorophore or an epitope label. When employing a number, n, of different pathogen-specific probes or probe sets, preferably a same number, n, of different detectable labels is used, such that each pathogen-specific probe or probe set comprises a different detectable label.

In another embodiment, the one or more pathogen-specific probes or probe sets are immobilized on a solid substrate comprising a plurality of identifying positions, each of the one or more pathogen-specific probes or probe sets occupying one of the plurality of identifying positions. The solid substrate can comprise silicon, glass, plastic, poylacrylamide, a polymer matrix, an agarose gel, a polyacrylamide gel, an organic membrane, or an inorganic membrane.

The present invention further provides a microarray for detecting a pathogen in a biological sample. As disclosed herein, the microarray comprises: (a) a solid support having a plurality of identifying positions; and (b) a plurality of pathogen-specific probe sets, each probe set occupying an identifying position on the solid support. The solid support can comprise silicon, glass, plastic, polyacrylamide, a polymer matrix, an agarose gel, a polyacrylamide gel, an organic porous membrane, or an inorganic porous membrane.

Preferably, each of the pathogen-specific probe sets comprising a plurality of pathogen-specific probe sets comprises two or more pathogen-specific probes. The disclosed microarray can optionally be constructed such that each of the two or more pathogen-specific probes of a pathogen-specific probe set comprises a different nucleotide sequence of a same pathogen. Preferably, each of the two or more pathogen-specific probes of a pathogen-specific probe set comprises at least about four or five pathogen-specific probes, or more preferably about 6 to 10 pathogen-specific probes, each pathogen-specific probe comprising a different nucleotide sequence of a same pathogen.

A microarray of the present invention preferably comprises a pathogen-specific probe set wherein each of the two or more pathogen-specific probes comprises a nucleotide sequence derived from a bacterium, a fungus, a virus, a protozoan, or a parasite. Optionally, a pathogen-specific probe can comprise a nucleotide sequence of a pathogen gene, preferably a nucleotide sequence encoding a pathogen polypeptide.

Accordingly, it is an object of the present invention to provide a method for detecting a pathogen in a biological sample and a microarray format for performing the method. The object is achieved in whole or in part by the present invention.

An object of the invention having been stated herein above, other objects will become evident as the description proceeds when taken in connection with the accompanying Examples and Figures as best described herein below.

### BRIEF DESCRIPTION OF THE FIGURES

FIG. 1 is an autoradiograph of a hybridization experiment as described in Example 13; and

FIGS. 2A and 2B are autoradiographs of a hybridization experiment as described in Example 14.

### DETAILED DESCRIPTION OF THE INVENTION

I. Biological Samples

The present invention provides methods that can be used to detect a pathogen in a biological sample. The term “biological sample” as used herein refers to a sample derived from a heterologous organism or from a heterogeneous composition. In each case, a biological sample is anticipated to represent a pathogen as well as a non-pathogenic organism or other biological matter.

The term “heterologous organism” indicates a non-pathogenic host organism, including any animal and any plant, samples therefrom, or parts thereof. The term “heterologous organism” encompasses both live and extant organisms. Thus, the term “heterologous organism” includes a blood sample intended for a clinical use such as transfusion as well as a dried blood sample or other forensic samples.

The term “heterogeneous composition” refers to a composition comprising a pathogen in population with any other live organism or extant organism, or part thereof. Representative heterogeneous compositions include a food sample or an environmental sample.

The present invention is directed toward detection assays wherein a pathogen to be detected comprises a subset of a sufficiently representative sample. The phrases “sufficiently representative” and “sufficiently large” each refer to a sample such that under-represented pathogens are not excluded from the sample. Statistical considerations and a suggested experimental approach relevant to representative sampling are discussed in Navidid et al. (1992) Am J Hum Genet 50:347-349. Briefly, a multiple-tubes procedure can be used, wherein a nucleic acid sample is divided among several tubes, amplified, then genotyped or otherwise analyzed. A statistical procedure is used to calculate a degree of certainly in the result.

I.A. Clinical Samples

A clinical sample, as used herein, refers to a sample derived from a subject. The term “subject” is desirably a human subject, although it is to be understood that the principles of the invention indicate that the invention is effective with respect to invertebrate and to all vertebrate species, including mammals, which are intended to be included in the term “subject”. Moreover, a mammal is understood to include any mammalian species in which detection of an infectious agent is desirable, particularly agricultural and domestic mammalian species. The methods of the present invention are particularly useful in the diagnosis of an infection in warm-blooded vertebrates, e.g., mammals and birds. The methods of the present invention can also be used to detect a pathogen in fish or other aquatic organisms, particularly those intended for consumption.

More particularly, the present invention can be used for detection of a pathogenic agent in a mammal such as a human. Also contemplated is detection of a pathogenic agent in mammals of importance due to being endangered (such as Siberian tigers), of economic importance (animals raised on farms for consumption by humans) and/or social importance (animals kept as pets or in zoos) to humans, for instance, carnivores other than humans (such as cats and dogs), swine (pigs, hogs, and wild boars), ruminants (such as cattle, oxen, sheep, giraffes, deer, goats, bison, and camels), and horses. Also contemplated is diagnosis of birds, including those kinds of birds that are endangered, or kept in zoos, as well as fowl, and more particularly domesticated fowl, i.e., poultry, such as turkeys, chickens, ducks, geese, guinea fowl, quail, pheasant, and the like, as they are also of economic importance to humans. Thus, contemplated is detection of a pathogen in livestock, including, but not limited to, domesticated swine (pigs and hogs), ruminants, poultry, and the like.

Fish represent a category of animals of interest for agricultural and ecological reasons. Representative fish species include, but are not limited to, trout, salmon, carp, shark, ray, flounder, sole, tilapia, medaka, goldfish, guppy, molly, platyfish, swordtail, zebrafish, loach, catfish, and the like.

A clinical specimen can be a blood sample; plasma; urine; stool; sputum; a biopsy; cerebrospinal fluid; a lesional or ulcerous swab; pus; a throat, nose, or nasopharyngeal swab; mucus, an endotracheal aspirate; bronchoalveolar lavage; a conjuctival or corneal swab; bone marrow; skin; hair, nails; any other clinical sample, or combinations thereof to create a composite sample. It is also considered an aspect of the invention to enrich a pathogen present in any of the above-mentioned samples by culturing or otherwise promoting the growth of the pathogen in the sample. Protocols for preparation of clinical samples are known in the art and can be found, for example, in Quinn (1997) in Lee et al., eds., Nucleic Acid Amplification Technologies: Application to Disease Diagnostics, pp.49-60, Birkhäuser Boston, Cambridge, Mass., United States of America; Richardson & Warnock (1993) Fungal Infection: Diagnosis and Management, Blackwell Scientific Publications Inc., Boston, Mass., United States of America; Storch (2000) Essentials of Diagnostic Virology, Churchill Livingstone, New York, N.Y.; Fisher & Cook (1998) Fundamentals of Diagnostic Mycology, W. B. Saunders Company, Philadelphia, Pa.; and White & Fenner (1994) Medical Virology, 4^{th} Edition, Academic Press, San Diego, Calif.

I.B. Plants

The term “a plant”, as used herein refers to an entire plant as well as the individual parts thereof, including but not limited to seeds, leaves, stems, and roots, as well as plant tissue cultures.

The disclosed methods can be used to detect a pathogen derived from a plant, and are particularly relevant for detection of a pathogen in a plant of agricultural importance or other economic importance, such as turfs and ornamentals. Representative agricultural plants include but are not limited to rice, wheat, barley, rye, corn, potato, carrot, sweet potato, sugar beet, bean, pea, chicory, lettuce, cabbage, cauliflower, broccoli, turnip, radish, spinach, asparagus, onion, garlic, eggplant, pepper, celery, carrot, squash, pumpkin, zucchini, cucumber, apple, pear, quince, melon, plum, cherry, peach, nectarine, apricot, strawberry, grape, raspberry, blackberry, pineapple, avocado, papaya, mango, banana, tobacco, tomato, sorghum and sugarcane. Methods for preparation of plant samples comprising nucleic acid material are known in the art, and representative protocols can be found, for example, in Guidet (1994) Nuc Acids Res 22(9):1772; Wang et al. (1993) Nuc Acids Res 21(17):4153; Dashek, ed. (1997) Methods in Plant Biochemistry and Molecular Biology, CRC Press, Boca Raton, Fla.; Maliga et al., eds. (1995) Methods in Plant Molecular Biology: A Laboratory Course Manual, Cold Spring Harbor Laboratory Press, Plainview, N.Y.; and Clark, ed. (1997) Plant Molecular Biology: A Laboratory Manual, Springer, New York, N.Y.

I.C. Food Samples

For detection of a pathogen in a biological sample comprising a food sample, a food matrix is homogenized, and extracted to remove potential inhibitors of subsequent enzymatic reactions. Nucleic acid molecules are then isolated from the food matrix. Representative methods for preparing nucleic acids from food samples for subsequent PCR analyses are known in the art and can be found, for example, in Giesendorf et al. (1992) Appl Environ Microbiol 58:3804-3808; Cano et al. (1993) J Appl Bacteriol 75:247-253; and Keasler & Hill (1992) J Food Protection 55:382-384. See also Barrett et al. (1997) in Lee et al., eds., Nucleic Acid Amplification Technologies: Application to Disease Diagnosis, pp. 171-182, Birkhäuser, Boston, Mass., United States of America, and references cited therein. Optionally, an enrichment step can be performed to assist recovery of bacteria that are sublethally stressed by physical or chemical processing as described by Feng (1992) J Food Protection 55:927-934, by Ray (1997) J Food Protection 42:346-355, and by Swaminathan & Feng (1994) Annu Rev Microbiol 48:401-426, including references cited therein. Food samples also include but are not limited to samples suspected of containing a pathogen associated with biological warfare or bioterrorism.

I.D. Environmental Samples

The term “environmental sample” as used herein refers to soil, water, sludge, or any other natural sample. Protocols for collecting, preserving, and handling environmental samples can be found, for example, in Csuros (1994) Environmental Sampling and Analysis for Technicians, Lewis Publishers, Boca Raton, Fla., United States of America. Techniques for extracting nucleic acids from environmental samples such that the nucleic acids can be used for subsequent amplification reactions are known in the art. See e.g., Bej et al. (1991) Appl Environ Microbiol 57:2429-2432, Tsai & Olson (1992) Appl Environ Microbiol 59:353-357, and Way et al. (1993) Appl Environ Microbiol 59:1473-1479. Environmental samples include but are not limited to samples suspected of containing a pathogen associated with biological warfare or bioterrorism.

II. Nucleic Acids

A biological sample that can be analyzed in accordance with the present invention comprises nucleic acid material. The terms “nucleic acid material”, “nucleic acids”, and “nucleic acid molecules” each refer to deoxyribonucleotides, ribonucleotides, and polymers and folded structures thereof in either single- or double-stranded form. Nucleic acids can be derived from any source, including any organism. Deoxyribonucleic acids can comprise genomic DNA, cDNA derived from ribonucleic acid, DNA from an organelle (e.g., mitochondrial DNA or chloroplast DNA), or combinations thereof. Ribonucleic acids can comprise genomic RNA (e.g., viral genomic RNA), messenger RNA (mRNA), ribosomal RNA (rRNA), transfer RNA (tRNA), or combinations thereof.

The biological sample can comprise nucleic acids from several organisms. For example, in the case of a host organism that has an infection, a biological sample derived from the host organism is prepared to include nucleic acids from both the host and the infectious agent. A biological sample can also comprise nucleic acids from a host and a plurality of infectious agents, or nucleic acids from an infectious agent in a population with other heterologous organisms. In each case, the proportion of nucleic acids derived from a pathogen comprises a subset of the nucleic acid material. The methods of the present invention employ non-selective amplification of the nucleic acid material such that pathogen-specific nucleic acids are elevated to a detectable level when used in subsequent hybridization assays.

The disclosed method for detection of a pathogen in a biological sample can be tailored to meet the needs of a particular diagnostic setting. For example, the disclosed method for detection of a pathogen in a biological sample can be tailored by varying the amount of a given nucleic acid sample. A representative approach for determining a suitable amount of an amplified target nucleic acid sample is set forth in Example 11.

II.A. Enrichment of Nucleic Acids

The present invention encompasses use of a sufficiently large biological sample to enable a comprehensive survey of low abundance nucleic acids in the sample. Thus, the sample can optionally be concentrated prior to isolation of nucleic acids. Several protocols for concentration have been developed that alternatively use slide supports (Kohsaka & Carson (1994) J Clin Lab Anal 8:425-455; Millar et al. (1995) Anal Biochem 226:325-330), filtration columns (Bej et al. (1991) Appl Environ Microbiol 57:3529-3534) or immunomagnetic beads (Albert et al. (1992) J Virol 66:5627-5630; Chiodi et al. (1992) J Clin Microbiol 30:255-258). Such approaches can significantly increase the sensitivity of subsequent detection methods.

As one example, SEPHADEX® matrix (Sigma of St. Louis, Mo., United States of America) is a matrix of diatomaceous earth and glass suspended in a solution of chaotropic agents and has been used to bind nucleic acid material (Boom et al. (1990) J Clin Microbiol 28:495-503; Buffone et al. (1991) Clin Chem 37:1945-1949). After the nucleic acid is bound to the solid support material, impurities and inhibitors are removed by washing and centrifugation, and the nucleic acid is then eluted into a standard buffer. Target capture also allows the target sample to be concentrated into a minimal volume, facilitating the automation and reproducibility of subsequent analyses (Lanciotti et al. (1992) J Clin Microbiol 30:545-551).

II.B. Nucleic Acid Isolation

Methods for nucleic acid isolation can comprise simultaneous isolation of total nucleic acid, or separate and/or sequential isolation of individual nucleic acid types (e.g., genomic DNA, cDNA, organelle DNA, genomic RNA, mRNA, polyA⁺ RNA, rRNA, tRNA) followed by optional combination of multiple nucleic acid types into a single sample.

When mRNA is selected as a biological sample, the disclosed method enables an assessment of pathogen gene expression. For example, detecting a pathogen in a biological sample can comprise determination of expressed virulence factors, other deleterious agents produced by a pathogen, or biosynthetic enzymes that generate virulence or other harmful pathogen gene products. Such analysis can facilitate distinction between active and latent infection and indicate severity of an infection.

Methods for nucleic acid isolation can be optimized to promote recovery of pathogen-specific nucleic acids. In some organisms, for example fungi, protozoa, gram-positive bacteria, and acid-fast bacteria, cell lysis and nucleic acid release can be difficult to achieve using general procedures, and therefore a method can be chosen that creates minimal loss of the pathogen subset of the sample.

RNA isolation methods are known to one of skill in the art. See Albert et al. (1992) J Virol 66:5627-2630; Busch et al. (1992) Transfusion 32:420-425; Hamel et al. (1995) J Clin Microbiol 33:287-291; Herrewegh et al. (1995) J Clin Microbiol 33:684-689; Izraeli et al. (1991) Nuc Acids Res 19:6051; McCaustland et al. (1991) J Virol Methods 35:331-342; Natarajan et al. (1994) PCR Methods Appl 3:346-350; Rupp et al. (1988) BioTechniques 6:56-60; Tanaka et al. (1994) J Gen Virol 75:2691-2698; and Vankerckhoven et al. (1994) J Clin Microbiol 30:750-753. A representative procedure for RNA isolation from a clinical sample is set forth in Example 1.

Methods for DNA isolation from infectious agents can employ a similar lysis protocol as described in Example 1, and the steps disclosed therein can be modified for purification of genomic DNA. See also Salamon et al. (2000) Genome Res 10(12):2044-2054; Gingeras et al. (1998) Genome Res 8:435-448; and Winzeler et al. (1998) Science 281:1194.

Simple and semi-automated extraction methods can also be used for nucleic acid isolation, including for example, the SPLIT SECOND™ system (Boehringer Mannheim of Indianapolis, Ind., United States of America), the TRIZOL™ Reagent system (Life Technologies of Gaithersburg, Md., United States of America), and the FASTPREP™ system (Bio 101 of La Jolla, Calif., United States of America). See also Smith (1998) The Scientist 12(14):21-24; and Paladichuk (1999) The Scientist 13(16):20-23.

Preferably, nucleic acids that are used for subsequent amplification and labeling are analytically pure as determined by spectrophotometric measurements or by visual inspection following electrophoretic resolution. Also preferably the nucleic acid sample is free of contaminants such as polysaccharides, proteins and inhibitors of enzyme reactions. When an RNA sample is intended for use as probe, it is preferably free of DNAase and RNAase. Contaminants and inhibitors can be removed or substantially reduced using resins for DNA extraction (e.g., CHELEX™ 100 from BioRad Laboratories of Hercules, Calif., United States of America) or by standard phenol extraction and ethanol precipitation. Isolated nucleic acids can optionally be fragmented by restriction enzyme digestion or shearing prior to amplification.

In one embodiment, the purification method specifically depletes host nucleic acids from the biological sample, concomitantly increasing the proportion of pathogen-specific nucleic acids in the sample. For example, high-stringency hybridization can be performed using host-specific probes followed by subtraction of hybrids from the sample. U.S. Pat. No. 5,759,778 to Li et al. discloses a method for generating a haptenylated probe that can be isolated using a hapten ligand bound to a solid support. Such a method can be used to select and then discard host-specific nucleic acids from a sample. The remaining non-host nucleic acids are then analyzed using the disclosed method for detecting a pathogen in a biological sample.

II.C. Amplification of Nucleic Acids

The terms “template nucleic acid” and “target nucleic acid” as used herein each refer to nucleic acids isolated from a biological sample as described herein above. The terms “template nucleic acid pool”, “template pool”, “target nucleic acid pool”, and “target pool” each refer to an amplified sample of “template nucleic acid”. Thus, a target pool comprises amplicons generated by performing an amplification reaction using the template nucleic acid. Preferably, a target pool is amplified using a random amplification procedure as described herein.

The term “target-specific primer” refers to a primer that hybridizes selectively and predictably to a target sequence, for example a pathogen-specific sequence, in a target nucleic acid sample. A target-specific primer can be selected or synthesized to be complementary to known nucleotide sequences of target nucleic acids.

The term “random primer” refers to a primer having an arbitrary sequence. The nucleotide sequence of a random primer can be known, although such sequence is considered arbitrary in that it is not designed for complementarity to a nucleotide sequence of the pathogen-specific probe. The term “random primer” encompasses selection of an arbitrary sequence having increased probability to be efficiently utilized in an amplification reaction. For example, the Random Oligonucleotide Construction Kit (ROCK available from http://www.sru.edu/depts/artsci/bio/ROCK.htm) is a macro-based program that facilitates the generation and analysis of random oligonucleotide primers (Strain & Chmielewski (2001) BioTechniques 30(6):1286-1291). Representative primers include but are not limited to random hexamers and rapid amplification of polymorphic DNA (RAPD)-type primers as described by Williams et al. (1990) Nuc Acids Res 18(22):6531-6535.

A random primer can also be degenerate or partially degenerate as described by Telenius et al. (1992) Genomics 13:718-725. Briefly, degeneracy can be introduced by selection of alternate oligonucleotide sequences that can encode a same amino acid sequence.

In one embodiment, random primers can be prepared by shearing or digesting a portion of the template nucleic acid sample. Random primers so-constructed comprise a sample-specific set of random primers.

The term “heterologous primer” refers to a primer complementary to a sequence that has been introduced into the template nucleic acid pool. For example, a primer that is complementary to a linker or adaptor, as described below, is a heterologous primer. Representative heterologous primers can optionally include a poly(dT) primer, a poly(T) primer, or as appropriate, a poly(dA) or poly(A) primer.

The term “primer” as used herein refers to a contiguous sequence comprising preferably about 6 or more nucleotides, more preferably about 10-20 nucleotides (e.g. 15-mer), and even more preferably about 20-30 nucleotides (e.g. a 22-mer). Primers used to perform the method of the present invention encompass oligonucleotides of sufficient length and appropriate sequence so as to provide initiation of polymerization on a nucleic acid molecule.

The term “random amplification” refers to an amplification procedure wherein each of a pool of resulting amplicons is generated using at least one random primer (as defined herein) or at least one heterologous primer (as defined herein). Specifically, the disclosed method does not perform amplification using two target-specific primers (as defined herein). The term “random amplicons” as used herein refers to amplicons generated using a random amplification technique.

The term “random amplification”, as used herein, also encompasses semi-random amplification of nucleic acid material. When using semi-random amplification methods, amplicons can be amplified using a primer pair, the primer pair comprising: (a) one or more random primers and one target-specific primer per probe; or (b) one or more heterologous primers and one target-specific primer per probe. The resulting amplicon pool is semi-random in that amplicons within the pool are not generated using two target-specific primers. Semi-random amplification using a target-specific primer and a random/heterologous primer can potentially increase the number of amplicons representing pathogen-specific sequences in the resulting target pool.

Thus, depending on primer selection, random amplification of a nucleic acid sample can comprise: (a) amplifying each potential target sequence in the nucleic acid sample such that the resulting pool of amplicons is a linearly more populous derivative of the original sample; or (b) amplifying a representative random subset of the target nucleic acids (e.g., when amplification is performed using a single random primer). Amplicons generated by any random or semi-random amplification method can further be combined as a sample pool that is hybridized with one or more pathogen-specific probes.

In one embodiment, primers selected for random amplification of the nucleic acid sample are also employed for probe preparation, as described further herein below. For example, one or more random primers can be used to amplify the nucleic acids of a biological sample. The same one or more random primers can also be used to amplify pure pathogen-specific nucleic acids, and the resulting pathogen-specific amplicons, or subset thereof, are used as probes. Subsequent hybridization of a target pool and probes generated using a same random primer or set of primers can be expected to enhance the sensitivity of the present inventive method.

Random amplification of the nucleic acid material can be accomplished by any effective method, including but not limited to random-primed PCR (RP-PCR), Degenerate Oligonucleotide Primed PCR (DOP-PCR), Primer-Extension Pre-amplification (PEP), any one of several related techniques that employ a nucleic acid linker or adaptor, Whole Genome PCR, and Transcription-Based Amplification, each technique described further herein below. Several procedures have been developed specifically for random amplification of RNA, including but not limited to Amplified Antisense RNA (aRNA) and Global RNA Amplification, also described further herein below.

Random-Primed PCR (RP-PCR). A sample of genomic DNA can be amplified using random hexamers. A representative embodiment of this approach is described in Example 2. This technique is referred to as random-primed polymerase chain reaction (RP-PCR). Briefly, a two-phase procedure is employed to achieve both high fidelity and high yield. In the initial phase, the amount of thermostable DNA polymerase, such as Taq polymerase, and dNTPs is sufficiently low in order to restrict errors caused by the enzyme. Also, the initial phase uses an extension temperature that supports a good combination of fidelity and efficiency of DNA polymerase activity (McPherson et al. (1995) PCR 2: A Practical Approach, IRL Press, New York, N.Y.), and is performed in a minimal volume to facilitate precise temperature control. See Peng et al. (1994) J Clin Pathol 47:605-608.

The RP-PCR procedure can increase the sensitivity of subsequent PCR using gene-specific primers approximately 100-fold by increasing the amount of template DNA (Peng et al. (1994) J Clin Pathol 47:605-608). Further, a survey of multiple genes indicates that RP-PCR generates a representative template pool (Peng et al. (1994) J Clin Pathol 47:605-608).

A similar procedure employs 22-mer primers having partially degenerate sequence and a dual annealing step for random amplification. See Telenius et al. (1992) Genomics 13:718-725.

As used herein, random-primed PCR also encompasses random amplification using a single random primer, for example a RAPD-type primer comprising an arbitrary sequence of about 10 nucleotides as described in Example 3 (Williams et al. (1990) Nuc Acids Res 18(22):6531-6535). More preferably, two RAPD-type primers can be used (Hopkins & Hilton (2001) BioTechniques 30(6):1262-1267).

Linker/Adaptor-based DNA Amplification. Several amplification methods have been developed that use an oligonucleotide linker/adaptor capable of random attachment to target DNA sequences, followed by amplification of the target sequence using primers complementary to the linker/adaptor. For example, Sequence-Independent, Single-Primer Amplification (SISPA) employs a primer having a sequence complementary to an adapter sequence that is ligated to the target DNA to amplify and thereby facilitate cloning and recovery of low-abundance genetic sequences. See Reyes & Kim (1991) Mol Cell Probes 5:473-481.

A related method employs a poly(dT) adapter sequence that can be added to target DNA using a standard terminal deoxytransferase reaction. The poly(dT)-tailed ends can then be used as primer binding sites for the complementary homooligonucleotides (Tam et al. (1989) FASEB J 3:1626).

Another variation of this technique incorporates a ligation step prior to amplification (Foo et al. (1992) Biotechniques 12(6):811-814). Briefly, blunt-ended, 5′-phosphorylated DNA fragments to be amplified are ligated to a large excess of unphosphorylated end-modifier. Non-phosphorylation of the end-modifier prevents its oligomerization at high concentration. The end-modifier is blunt on one end and has a 5′ overhang on the other end (e.g., a BamH I digested site). Upon ligation, the end-modifier is positioned in the same orientation at each end of the DNA fragment. See also Seth et al. (1986) Gene 42:49-57; Haymerle et al. (1986) Nuc Acids Res 14:8615-8624; and Mueller & Wold (1989) Science 246:780-785. PCR can be performed using the ligated DNA fragments as template and a single primer having the sequence of the 5′ overhang strand of the end-modifier, e.g. an about 18-20 base pair nucleotide sequence of the 5′ end of the end-modifier.

When used to amplify a population of DNA fragments isolated from ancient tissues (approximately 100-600 base pairs in size), the size range of fragments after amplification was similar to that observed in freshly extracted DNA, suggesting that the method yields a representative amplified population. Further, this method did not reveal evidence for formation of chimeric sequences due to complementarity of DNA fragment ends, as observed using alternate techniques (Lawlor et al. (1991) Nature 349:785-788).

Whole Genome PCR. This method is performed by ligating a blunt-ended and nonpalindromic catch linker to the blunt ends of DNA fragments, amplifying the target DNA using primers designed according to the catch linker sequence, and selecting the amplified DNA based on protein-DNA binding (Kinzler & Vogelstein (1989) Nuc Acids Res 17(10):3645-3653). Following two cycles of amplification and binding, this procedure can yield an approximately 1000-fold increase in the sensitivity of detecting a single nucleotide sequence, although smaller fragments may be preferentially amplified.

Primer-Extension Preamplification (PEP). This technique uses primer extension reactions to amplify a large percentage of DNA sequences present in a small sample such as a single cell. Multiple rounds of extension with Taq DNA polymerase and a random mixture of 15 base pair oligonucleotides as primers can produce multiple copies of DNA sequences originally present in the sample. At least 78% of the genomic sequences in human haploid cells are estimated to be copied a minimum of 30 times (95% confidence). See Zhang et al. (1992) Proc Natl Acad Sci USA 89:5847-5851.

Transcription-Based Amplification. Transcription-based amplification systems involve synthesizing a DNA molecule complementary to the target nucleic acid followed by in vitro transcription with the newly synthesized cDNA as a template (Kwoh et al. (1989) Proc Natl Acad Sci USA 86:1173-1177). This process is variously called self-sustaining sequence replication, nucleic acid sequence-based amplification (NASBA), or transcription-mediated amplification (TMA) (Podzorski et al. (1995) in Murray et al., eds., Manual of Clinical Microbiology, p.130, American Society for Microbiology, Washington, D.C.; Persing et al., eds. (1993) Diagnostic Molecular Microbiology—Principles and Applications, American Society for Microbiology, Washington, D.C.). Briefly, DNA is synthesized using reverse transcriptase and a primer having a bacterial phage T7 RNA polymerase-binding site. The amplicon is then transcribed using T7 RNA polymerase. Each of the two steps can be variably repeated. For random transcription-based amplification, a random primer having T7 RNA polymerase binding site is used. Amplified Antisense RNA, described herein below, is a transcription-based method for amplifying RNA.

Amplified Antisense RNA (aRNA). A population of RNA can be amplified using a technique referred to as Amplified Antisense RNA (aRNA) as described in Example 4. See Van Gelder et al. (1990) Proc Natl Acad Sci USA 87:1663-1667 and Wang et al. (2000) Nat Biotech 18(4):457-459. Briefly, an oligo(dT) primer is synthesized such that the 5′ end of the primer includes a T7 RNA polymerase promoter. This oligonucleotide can be used to prime the poly(A)⁺ mRNA population to generate cDNA. Following first strand cDNA synthesis, second strand cDNA is generated using RNA nicking and priming (Sambrook et al. (1989) Molecular Cloning: A Laboratory Manual, 2^{nd} Edition, Cold Spring Harbor Laboratory Press, Cold Spring Harbor, N.Y.). The resulting cDNA is treated briefly with S1 nuclease and blunt-ended with T4 DNA polymerase. The cDNA is then used as a template for transcription-based amplification using the T7 RNA polymerase promoter to direct RNA synthesis.

Eberwine et al. adapted the aRNA procedure for in situ random amplification of RNA followed by target-specific amplification. The successful amplification of under represented transcripts suggests that the pool of transcripts amplified by aRNA is representative of the initial mRNA population (Eberwine et al. (1992) Proc Natl Acad Sci USA 89:3010-3014).

Global RNA Amplification. U.S. Pat. No. 6,066,457 to Hampson et al. describes a method for substantially uniform amplification of a collection of single stranded nucleic acid molecules such as RNA. Briefly, the nucleic acid starting material is anchored and processed to produce a mixture of directional shorter random size DNA molecules suitable for amplification of the sample.

In accordance with the methods of the present invention, any one of the above-mentioned PCR techniques or related techniques can be employed to perform the step of amplifying the nucleic acid sample. In addition, such methods can be optimized for amplification of a particular subset of nucleic acid (e.g., genomic DNA versus RNA), and representative optimization criteria and related guidance can be found in the art. See Cha & Thilly (1993) PCR Methods Appl 3:S1 8-S29; Linz et al. (1990) J Clin Chem Clin Biochem 28:5-13; Robertson & Walsh-Weller (1998) Methods Mol Biol 98:121-154; Roux (1995) PCR Methods Appl 4:S185-S194; Williams (1989) BioTechniques 7:762-769; and McPherson et al. (1995) PCR 2: A Practical Approach, IRL Press, N.Y., N.Y.

II.D. Nucleic Acid Size and Secondary Structure

Optionally, randomly amplified nucleic acids of a biological sample can be fragmented to enhance subsequent hybridization efficiency, in part by minimizing secondary structure in target molecules. See Southern et al. (1999) Nat Genet Suppl 21:5-9 and Wodicka et al. (1997) Nat Biotechnol 15:1359-1366. Briefly, fragmentation approaches include restriction enzyme digests and shearing of the amplified template DNA.

Alternatively, a randomly amplified nucleic acid sample can be used to generate folded target nucleic acids having both double-stranded and single-stranded segments as described in U.S. Pat. No. 6,214,545 to Dong et al. Briefly, folded target DNAs are produced from either single-stranded or double-stranded target DNAs by denaturing the DNA and then permitting the DNA to form intra-strand secondary structure. The secondary structure can be referred to as a dendrimer. The target DNA can be denatured by any of a variety of methods known in the art including heating and exposure to alkali. Similarly, any one of several renaturing conditions that favor the formation of intra-strand duplexes can be used, for example, cooling or diluting the DNA solution, or neutralizing the pH of the DNA solution. A folded nucleic acid can be further hybridized with one or more oligonucleotide probes to form a folded nucleic acid/probe complex as described in U.S. Pat. No. 6,194,149 to Neri et al.

III. Pathogen-Specific Probes and Control Probes

To detect a pathogen in a biological sample, the methods of the present invention include a step of hybridizing randomly amplified nucleic acids derived from the biological sample to one or more pathogen-specific probes or probe sets. As described further herein below, pathogen-specific probes and probe sets can be synthesized or otherwise generated using nucleotide sequences from any potential pathogen.

For any particular application wherein a pathogen is detected in a biological sample using the methods of the present invention, probes or probe sets can optionally be selected as appropriate for the application. For example, for detection of a pathogen in a human, probes or probe sets can be selected to represent common and/or potential human pathogens. Similarly, for detection of a pathogen in a plant, probes or probe sets can be selected to represent common and/or potential plant pathogens. In a preferred embodiment, every known pathogen for any given host species is represented by one or more probes. Probes and probe sets can further be selected to distinguish among potential pathogen variants, as described further herein.

III.A. Pathogens

The term “pathogen”, whose presence is detected in accordance with the present invention, can be a bacterium, a virus, a fungus, a protozoan, a parasite, other infective agent, or potentially harmful or parasitic organism as would be apparent to one of ordinary skill in the art of a review of the present disclosure.

Representative bacteria related to clinical conditions and that are detected by the disclosed methods include but are not limited to species of the genera Salmonella, Shigella, Actinobacillus, Porphyromonas, Staphylococcus, Bordetella, Yersinia, Haemophilus, Streptococcus, Chlamydophila, Alliococcus, Campylobacter, Actinomyces, Neisseria, Chlamydia, Treponema, Ureaplasma, Mycoplasma, Mycobacterium, Bartonella, Legionella, Ehrlichia, Escherichia, Listeria, Vibrio, Clostridium, Tropheryma, Actinomadura, Nocardia, Streptomyces, and Spirochaeta.

Viruses that can be detected in accordance with the present invention include DNA viruses, such as Poxviridae, Herpesviridae, Adenoviridae, Papoviridae, Hepadnaviridae, and Parvoviridae. RNA viruses are also envisioned to be detected in accordance with the disclosed methods, including Paramyxoviridae, Orthomyxoviridae, Coronaviridae, Arenaviridae, Retroviridae, Reoviridae, Picomaviridae, Caliciviridae, Rhabdoviridae, Togaviridae, Flaviviridae, and Bunyaviridae.

Representative viruses include but are not limited to, hepatitis viruses, flaviviruses, gastroenteritis viruses, hantaviruses, Lassa virus, Lyssavirus, picornaviruses, polioviruses, enteroviruses, nonpolio enteroviruses, rhinoviruses, astroviruses, rubella virus, HIV-1 (human immunodeficiency virus type 1), HIV-2 (human immunodeficiency virus type 2), HTLV-1 (human T-lymphotropic virus type 1), HTLV-2 (human T-lymphotropic virus type 2), HSV-1 (herpes simplex virus type 1), HSV-2 (herpes simplex virus type 2), VZV (varicellar-zoster virus), CMV (cytomegalovirus), HHV-6 (human herpes virus type 6), HHV-7 (human herpes virus type 7), EBV (Epstein-Barr virus), influenza A and B viruses, adenoviruses, RSV (respiratory syncytial virus), PIV-1 (parainfluenza virus, types 1, 2, and 3), papillomavirus, JC virus, polyomaviruses, BK virus, filoviruses, coltiviruses, orbiviruses, orthoreoviruses, retroviruses, and spumaviruses.

Representative fungi that infect animals, including humans, and that can be detected in a biological sample using the methods of the present invention include species of the genera Aspergillus, Trichophyton, Microsporum, Epidermaophyton, Candida, Malassezia, Pityrosporum, Trichosporon, Exophiala, Cladosporium, Hendersonula, Scytalidium, Piedraia, Scopulariopis, Acremonium, Fusarium, Curvularia, Penicillium, Absidia, Pseudallescheria, Rhizopus, Cryptococcus, MuCunninghamella, Rhizomucor, Saksenaea, Blastomyces, Coccidioides, Histoplasma, Paraoccidioides, Phialophora, Fonsecaea, Rhinocladiella, Conidiobolu, Loboa, Leptosphaeria, Madurella, Neotestudina, Pyrenochaeta, Colletotrichum, Alternaria, Bipolaris, Exserohilum, Phialophora, Xylohypha, Scedosporium, Rhinosporidium, and Sporothrix.

Fungal pathogens that infect plants and that can be detected in a biological sample using the methods of the present invention include but are not limited to Botrytis cinerea (grey mold fungus), Cladosporium fulvum (causative agent of leaf mold of tomato), Cochliobolus heterostrophus (causative agent of Southern Corn Leaf Blight), Colletotrichum (causative agent of leaf blight), Magnaportha grisea (rice blast fungus), Phytophthora (causative agent of late blight), Polymyxa graminis (causative agent of rust disease), Polymyxa distincta (causative agent of rust disease), Ustilago maydis (causative agent of corn smut disease), Rhizoctonia (causative agents of powdery and downy mildews), and species of the genera Candida, Cryptococcus; Fusarium, Ophiostoma, Rhyncosporium, Aspergillus, Rhizotonia, Phythium, Clariceps, Sclerotinia, Sclerotium, Acremonium, Fusiform, and Cryphonectria.

The detection method disclosed herein can also be used to detect viruses that infect plants, for example Tomato Spotted Wilt Virus and Tobacco Mosaic Virus. The present invention is also useful for detection of bacterial pathogens that infect plants including but not limited to Xanthomonas, Pseudomonas, Phytoplasma, and Ralstonia.

The term “pathogen” also encompasses parasites, such as species of the genera Rickettsiae; protozoan species of the genera Toxoplasma, Giardia, Cryptosporidium, Trichomonas, and Leishmania; and nematodes such as species of the genera Trichinella and Anisakis.

III.B. Probes

To detect a pathogen in a biological sample, the present invention provides that a nucleic acid molecule in a pool of randomly amplified nucleic acids of the biological sample specifically or substantially hybridizes to a pathogen-specific probe or probe set under relatively stringent conditions.

The term “probe” indicates a nucleic acid molecule having a capacity to selectively or substantially hybridize to a complementary nucleotide sequence in a heterogeneous mixture of nucleic acid molecules, as described further herein below. Additional methods for predicting and/or determining the specificity and selectivity of a probe can be found in International Publication No. WO 01/06013.

The term “complementary sequences”, as used herein, indicates two nucleotide sequences that comprise antiparallel nucleotide sequences capable of pairing with one another upon formation of hydrogen bonds between base pairs. As used herein, the term “complementary sequences” means nucleotide sequences which are substantially complementary, as can be assessed by hybridization to the nucleic acid segment in question under relatively stringent conditions such as those described herein. The term “complementary sequence” also includes a pair of nucleotides that bind a same target nucleic acid and participate in the formation of a triplex structure as described, for example in U.S. Pat. No. 6,027,893 to Ørum et al.

The term “probe set” refers to a collection of two or more probes, each probe having a nucleotide sequence that is substantially different than a nucleotide sequence of other probes in the probe set. The term “substantially different” in the context of nucleotide sequence comparisons refers to sequences that do not selectively or substantially hybridize to each other under relatively stringent hybridization conditions as described herein.

A preferred nucleotide sequence employed for hybridization includes probe sequences that are complementary to or mimic at least an approximately 14- to 40-nucleotide sequence of a nucleic acid molecule of a pathogen. Preferably, a probe comprises 14 to 20 nucleotides, more preferably about 20 to 40 nucleotides, or even longer where desired, such as 50, 60, 100, 200, 300, 500, or 1000 nucleotides, or up to the full length of any of a pathogen gene.

A probe used in accordance with the present invention can comprise DNA, RNA, PNA, and combinations thereof. A probe can further comprise modified nucleotides or universal base and can comprise a folded nucleic acid, such as a hairpin structure.

The term “pathogen-specific probe” indicates a nucleic acid molecule having a capacity to specifically recognize a pathogen sequence, including intergenic sequences. A pathogen-specific probe can further comprise a pathogen gene, and more preferably a nucleotide sequence that encodes a pathogen polypeptide. For example, a pathogen-specific probe can comprise a nucleotide sequence that encodes a polypeptide that mediates disease progression, i.e. toxic shock syndrome toxin-1 or an enterotoxin. As another example, a pathogen-specific probe can comprise a nucleotide sequence that encodes an enzyme in the biosynthetic pathway (which typically involves at least seventeen steps, and possibly more steps in some cases) for the production of aflatoxin, as described in Example 9.

The term “pathogen-specific probe set” indicates a collection of pathogen-specific probes, each pathogen-specific probe having a nucleotide sequence that is substantially different when compared with the nucleotide sequences of other probes comprising the probe set, and wherein each of the pathogen-specific probes specifically hybridizes to a nucleotide sequence from a same pathogen. Preferably, a pathogen-specific probe set comprises two or more pathogen-specific probes, more preferably about 3, 4, or 5 pathogen-specific probes, and more preferably about 6, 7, 8, 9, or 10 pathogen-specific probes. The use of a pathogen-specific probe set in accordance with the methods of the present invention is anticipated to improve sensitivity and reliability of detection of a pathogen.

A voluminous resource for pathogen-specific probes is available, and any such sequence that can specifically detect a pathogen can be employed to perform the methods of the present invention. The present invention further provides that the probes from a same pathogen can be combined as a probe set for use in accordance with the detection methods disclosed herein. Pathogen-specific probes can be designed according to nucleotide sequences in public sequence repositories (e.g., Sanger Centre (ftp://ftp.sanger.ac.uk/pub/tb/sequences) and GenBank (http://ncbi.nlm.nih.gov)), including cDNAs, ESTs, STSs, repetitive sequences, and genomic sequences. Complete genome sequences of 30 microbial species have been determined, and the current pace of research predicts that the complete genome sequences of more than 100 additional microbial species will be available in the next 2-4 years. See Fraser et al. (2000) Nature 406(17):799-803.

In the case where gene-specific probes are sought, RNA and protein modeling algorithms can be used to define expressed sequences. The term “gene” refers broadly to any segment of DNA associated with a biological function. A gene encompasses sequences including but not limited to a coding sequence, a promoter region, a cis-regulatory sequence, a non-expressed DNA segment that is a specific recognition sequence, for regulatory proteins, a non-expressed DNA segment that contributes to gene expression, or combinations thereof. Computational methods using Markov modeling techniques can now routinely predict greater than 99% of protein coding regions in bacterial genomes.

Numerous pathogen-specific sequences are known and can be used in accordance with the present invention. In one embodiment, pathogen-specific probes can be derived from available PCR diagnostic tests that employ target-specific primers to amplify a pathogen-specific sequence. Representative examples of pathogen-specific probes that have been used for related although distinct methods are summarized below. See also De Saizeu et al. (1998) Nature Biotechnol 16:45-48; U.S. Pat. Nos. 6,197,514 and 6,165,721; and International Publication No. WO 93/03186.

Mycobacterium tuberculosis. A microarray was assembled having 250-1000 base pair fragments representing nearly all open reading frames of the virulent M. tuberculosis strain H37Rv (Behr et al. (1999) Science 284:1479-1480). Comparative genomic analysis between M. tuberculosis and Mycobacterium bovis identified 11 genomic regions (encompassing 91 open reading frames) that are present in H37Rv but absent from one or more M. bovis strains (Behr et al. (1999) Science 284:1479-1480). The nucleotide sequences that lie in these genomic regions can be employed as probes for detection of virulent M. tuberculosis.

HIV. In another case, HIV-1 pol sequences were used to construct a microarray for detection of HIV-1 drug-resistant strains in human patients (Günthard et al. (1998) AIDS Res Hum Retroviruses 14:869-876). In contrast to the present invention, Günthard et al. employ nucleic acid samples that have been amplified using HIV-specific primers; however, the hybridization step of the present invention can utilize the HIV-1 probes described therein.

E. coli. Microarrays were assembled by immobilizing E. coli nucleic acids comprising the full-length sequence of each of 4290 open reading frames identified in the E. coli genome. Microarrays so constructed were used to study gene expression changes in response to heat shock and IPTG (isopropyl-β-D-thiogalactopyranoside) treatments (Richmond et al. (1999) Nuc Acids Res 27:3821-3835), and to describe gene regulation in rich versus minimal media growth conditions (Tao et al. (1999) J Bacteriol 181:6425-6440).

Pathogen-specific probes can also be generated using random or semi-random amplification techniques such that prior knowledge of a pathogen sequence is not required. For example, a random RAPD-type primer can be used to amplify a subset of sequences from a pure template nucleic acid sample derived from a pathogen as described in Example 5 (e.g., according to the method in Williams et al. (1990) Nuc Acids Res 18(22):6531-6535). The resulting random pathogen-specific amplicons can be used as probes according to the method disclosed herein. A pathogen-specific probe can also be amplified by semi-random amplification techniques, for example by using one random primer and one pathogen-specific primer.

When probes are prepared using random or semi-random amplification techniques, nucleic acids from a biological sample to be analyzed are preferably amplified using a same random primer and a same random or semi-random amplification technique. Subsequent hybridization of probes and an amplified nucleic acid sample, each generated using a same random primer, can be expected to increase sensitivity of the method of the present invention.

In another embodiment, primers used to amplify pathogen-specific sequences for use as probes can be prepared by shearing or digestion of pure pathogen-specific nucleic acids. A target nucleic acid sample to be hybridized with probes so prepared is optionally randomly amplified using primers that are prepared by similar shearing techniques or by digestion using a same restriction endonuclease of the target nucleic acid sample. Indeed, the approach can be taken with respect to the target nucleic acid sample, independent of the approach taken to obtain probes.

In some cases, a pathogen-specific probe can identify multiple pathogens, for example all or substantially all related subspecies of a particular species. See e.g., Liu et al. (2000) J Med Microbiol 49(6):493-497. Such a probe can be determined empirically as a probe showing substantial hybridization with multiple pathogens. Alternatively, a probe for recognition of multiple pathogens can be designed as complementary to a sequence that is identical or substantially identical in multiple pathogens.

The term “substantially identical” in regards to a nucleotide or polypeptide sequence means that a particular sequence varies from the sequence of a naturally occurring sequence by one or more deletions, substitutions, or additions. The term “substantially identical”, in the context of two nucleotide sequences, refers to two or more sequences or subsequences that have at least 60%, preferably about 70%, more preferably about 80%, more preferably about 90-95%, and most preferably about 99% nucleotide identity, when compared and aligned for maximum correspondence, as measured using one of the sequence comparison algorithms described herein below by visual inspection. In one aspect, polymorphic sequences can be substantially identical sequences. The term “polymorphic” refers to the occurrence of two or more genetically determined alternative sequences or alleles in a population. An allelic difference can be as small as one base pair.

For sequence comparison, typically one sequence acts as a reference sequence to which test sequences are compared. When using a sequence comparison algorithm, test and reference sequences are entered into a computer program, subsequence coordinates are designated if necessary, and sequence algorithm program parameters are selected. The sequence comparison algorithm then calculates the percent sequence identity for the designated test sequence(s) relative to the reference sequence, based on the selected program parameters.

Optimal alignment of sequences for comparison can be conducted, e.g., by the local homology algorithm of Smith & Waterman (1981) Adv Appl Math 2:482, by the homology alignment algorithm of Needleman & Wunsch (1970) J Mol Biol 48:443, by the search for similarity method of Pearson & Lipman (1988) Proc Natl Acad Sci USA 85:2444-2448, by computerized implementations of these algorithms (GAP, BESTFIT, FASTA, and TFASTA in the Wisconsin Genetics Software Package, Genetics Computer Group, Madison, Wis.), or by visual inspection. See generally, Ausubel et al. (1992) Current Protocols in Molecular Biology, John Wiley & Sons, Inc., New York, N.Y. A preferred algorithm for determining percent sequence identity and sequence similarity is the BLAST algorithm, which is described in Altschul et al. (1990) J Mol Biol 215: 403-410. Software for performing BLAST analyses is publicly available through the National Center for Biotechnology Information (http://www.ncbi.nlm.nih.gov/).

The BLAST algorithm also performs a statistical analysis of the similarity between two sequences. See e.g., Karlin & Altschul (1993) Proc Natl Acad Sci USA 90:5873-5887. One measure of similarity provided by the BLAST algorithm is the smallest sum probability (P(N)), which provides an indication of the probability by which a match between two nucleotide or amino acid sequences would occur by chance. For example, a test nucleic acid sequence is considered similar to a reference sequence if the smallest sum probability in a comparison of the test nucleic acid sequence to the reference nucleic acid sequence is less than about 0.1, more preferably less than about 0.01, and most preferably less than about 0.001.

Nucleotide sequence comparison techniques can also be used to identify candidate probes as substantially identical to numerous sequences not unique to a pathogen, for example host-specific sequences. This analysis can facilitate elimination of such probes from hybridization assays to avoid potential nonspecific hybridization.

Thus, the method of the present invention includes the use of probes that substantially hybridize to a target nucleic acid. The phrase “hybridizing substantially to” refers to complementary hybridization between a probe nucleic acid molecule and a target nucleic acid molecule and embraces hybridization of substantially identical sequences that can be accommodated by adjusting the stringency of the hybridization media to achieve the desired hybridization.

A pathogen-specific probe can further identify an individual pathogen to the exclusion of related species or variants of a single species. Treatment-resistant mutations have been described for a number of pathogens, and probes comprising a mutant sequence can be used to determine the presence of a treatment-resistant variant. In this case, the target nucleic acids are hybridized with pathogen-specific probes under relatively high stringency conditions as described further herein below. For example, hepatitis C genotype has been implicated as a predictor of response to interferon therapy and disease severity. The nucleotide sequence of the hepatitis C genome is heterogeneous, similar to that of human immunodeficiency virus. Six major genotypes (types 1 through 6) and several subtypes have been identified. More than 90% of hepatitis C infections in the United States are caused by genotypes 1-3 (Forns & Bukh (1998) Viral Hepatitis 4:1-19), although infection with genotype 1b is associated with poor response to interferon therapy (Davis & Lau (1997) Hepatology 26:122S-127S). Similarly, benzimadazole resistance in parasitic nematodes of sheep is correlated with loss of β-tubulin isotype 1 and 2 alleles (Roos et al. (1995) Parasitol Today 11:148-150). Thus, detection of pathogen variants can be useful for predicting the efficacy of treatment or amelioration of the infectious agent. See also Taylor et al. (2001) Biotechniques 30(3):661-669, Ye et al. (2001) Hum Mutat 17(4):305-316, Niblett et al. (2000) Virus Res 71(1-2):97-106, Günthard et al. (1998) AIDS Res Hum Retroviruses 14:869-876.

The disclosed method for detection of a pathogen in a biological sample can be tailored to meet the needs of a particular diagnostic setting. For example, the disclosed method for detection of a pathogen in a biological sample can be tailored by selection of an appropriate number of pathogen-specific probes. Furthermore, a representative approach for determining a suitable amount of a pathogen-specific probe is set forth in Example 10. The amount of probe required can also depend on whether a microarray is employed, or on whether a dot blot or slot blot technique is employed. The latter techniques, and techniques similar to them, are also referred to in the art as “macroarrays”.

The methods of the present invention also provide for the use of non-pathogen probes and probe sets that are positive controls for sample integrity. For example, a probe set can be designed complementary to host-specific sequences. Preferably, multiple positive control probes or probe sets are included in each assay to represent diverse probe features such as abundance of a complementary transcript (e.g. a relative amount of amplified target nucleic acid), probe stability, and probe guanine/cytosine (GC) content.

Optionally, the present invention can further comprise spotting total genomic DNA as a control, or even as a diagnostic. For example, labeled Cercospora nicotinae nucleic acids hybridize more strongly to spotted, genomic Cercospora nicotinae than to spotted, genomic Aspergillus flavus, Fusarium verticilliodes, Magnaportha grisea and others as well.

Preferably, an assay of the present invention also employs probes or probe sets to detect non-specific hybridization. Such a negative control probe or probe set can be derived from non-pathogenic and non-host nucleotide sequences. For example, a negative control probe can comprise sequences that are derived from a source heterologous to the biological sample being tested and that are not predicted to be present in the biological sample being tested. In the case of a mammalian subject, a suitable negative control probe can comprise a plant-specific sequence. Hybridization of the test nucleic acid sample to a negative control probe can indicate a level of background hybridization. Ideally, the test nucleic acid sample hybridizes to few, if any, such negative control probes.

For generation of pathogen-specific probes and control probes, relevant nucleic acid sequences can be cloned, synthesized, recombinantly altered, mutagenized, or combinations thereof. Preferably, the nucleotide sequence of a pathogen-specific probe generated by any of the above-mentioned techniques is determined prior to hybridization with a randomly-amplified nucleic acid sample. Standard molecular cloning and sequencing techniques are known in the art, and exemplary, non-limiting methods are described by Sambrook et al., eds. (1989) Molecular Cloning, Cold Spring Harbor Laboratory Press, Cold Spring Harbor, N.Y.; by Silhavy et al. (1984) Experiments with Gene Fusions, Cold Spring Harbor Laboratory Press, Cold Spring Harbor, N.Y.; by Ausubel et al. (1992) Current Protocols in Molecular Biology, John Wiley and Sons Inc., New York, N.Y.; and by Glover, ed. (1985) DNA Cloning: A Practical Approach, MRL Press Ltd., Oxford, United Kingdom.

IV. Nucleic Acid Labeling

Optionally, a randomly amplified sample or a pathogen-specific probe/probe set further comprises a detectable label. In one embodiment of the invention, the amplified nucleic acids can be labeled prior to hybridization with a set of probes. Alternatively, randomly amplified nucleic acids are hybridized with a set of probes, without prior labeling of the amplified nucleic acids. For example, an unlabeled pathogen-specific nucleic acid in the biological sample can be detected by hybridization to a labeled probe. In another embodiment, both the randomly amplified nucleic acids and the one or more pathogen-specific probes include a label, wherein the proximity of the labels following hybridization enables detection. An exemplary procedure using nucleic acids labeled with chromophores and fluorophores to generate detectable photonic structures is described in U.S. Pat. No. 6,162,603 to Heller.

In accordance with the methods of the present invention, the amplified nucleic acids or pathogen-specific probes/probe sets can be labeled using any detectable label. It will be understood to one of skill in the art that any suitable method for labeling can be used, and no particular detectable label or technique for labeling should be construed as a limitation of the disclosed methods.

Direct labeling techniques include incorporation of radioisotopic or fluorescent nucleotide analogues into nucleic acids by enzymatic synthesis in the presence of labeled nucleotides or labeled PCR primers. A radio-isotopic label can be detected using autoradiography or phosphorimaging. A fluorescent label can be detected directly using emission and absorbance spectra that are appropriate for the particular label used. Any detectable fluorescent dye can be used, including but not limited to FITC (fluorescein isothiocyanate), FLUOR X™, ALEXA FLUOR® 488, OREGON GREEN® 488, 6-JOE (6-carboxy-4′,5′-dichloro-2′, 7′-dimethoxyfluorescein, succinimidyl ester), ALEXA FLUOR® 532, Cy3, ALEXA FLUOR® 546, TMR (tetramethylrhodamine), ALEXA FLUOR® 568, ROX (X-rhodamine), ALEXA FLUOR® 594, TEXAS RED®, BODIPY® 630/650, and Cy5 (available from Amersham Pharmacia Biotech of Piscataway, N.J., United States of America or from Molecular Probes Inc. of Eugene, Oreg., United States of America). Fluorescent tags also include sulfonated cyanine dyes (available from Li-Cor, Inc. of Lincoln, Nebr., United States of America) that can be detected using infrared imaging. Methods for direct labeling of a heterogeneous nucleic acid sample are known in the art and representative protocols can be found in, for example, DeRisi et al. (1996) Nat Genet 14:457-460; Sapolsky & Lipshutz (1996) Genomics 33:445-456; Schena et al. (1995) Science 270:467-470; Schena et al. (1996) Proc Natl Acad Sci USA 93:10614-10619; Shalon et al. (1996) Genome Res 6:639-645; Shoemaker et al. (1996) Nat Genet 14:450-456; and Wang et al. (1998) Proc Natl Acad Sci USA 86:9717-9721. A representative procedure is set forth herein as Example 6.

Indirect labeling techniques can also be used in accordance with the methods of the present invention, and in some cases, can facilitate detection of rare target sequences by amplifying the label during the detection step. Indirect labeling involves incorporation of epitopes, including recognition sites for restriction endonucleases, into amplified nucleic acids prior to hybridization with the set of probes. Following hybridization, a protein that binds the epitope is used to detect the epitope tag.

In one embodiment, a biotinylated nucleotide can be included in the amplification reactions to produce a biotin-labeled nucleic acid sample. Following hybridization of the biotin-labeled sample with pathogen-specific probes as described herein below, the label can be detected by binding of an avidin-conjugated fluorophore, for example streptavidin-phycoerythrin, to the biotin label. Alternatively, the label can be detected by binding of an avidin-horseradish peroxidase (HRP) streptavidin conjugate, followed by colorimetric detection of an HRP enzymatic product.

In another embodiment, indirect labeling can comprise reporter deposition techniques, such as Tyramide Signal Amplification (TSA)™. For example, cDNA can be labeled using a biotinylated nucleotide, and HRP is then bound to the microarray using streptavidin linked to HRP. In the presence of hydrogen peroxide, HRP oxidizes the phenolic ring of tyramide conjugates (i.e., a cyanine-tyramide molecule) to produce highly reactive, free radical intermediates. These activated substrates subsequently form covalent bonds with tyrosine residues of nearby protein molecules used to block the microarray surface. HRP-catalyzed substrate conversion results in multiple depositions at the position of the hybridized probe. Typically TSA™ labeling can increase detection sensitivity approximately 50- to 100-fold compared to direct labeling methods. See also Bobrow et al. (1989) J Immunol Methods 125:279-285 and U.S. Pat. Nos. 5,731,158; 5,583,001; and 5,196,306.

The quality of probe or nucleic acid sample labeling can be approximated by determining the specific activity of label incorporation. For example, in the case of a fluorescent label, the specific activity of incorporation can be determined by the absorbance at 260 nm and 550 nm (for Cy3) or 650 nm (for Cy5) using published extinction coefficients (Randolph & Waggoner (1995) Nuc Acids Res 25:2923-2929). Very high label incorporation (specific activities of >1 fluorescent molecule/20 nucleotides) can result in a decreased hybridization signal compared with probe with lower label incorporation. Very low specific activity (<1 fluorescent molecule/100 nucleotides) can give unacceptably low hybridization signals. See Worley et al. (2000) in Shena, ed., Microarray Biochip Technology, pp. 65-86, Eaton Publishing, Natick, Mass., United States of America. Thus, it will be understood to one of skill in the art that labeling methods can be optimized for performance in microarray hybridization assay, and that optimal labeling can be unique to each label type.

V. Microarrays

In a preferred embodiment of the invention, the pathogen-specific probes or probe sets are immobilized on a solid support such that a position on the support identifies a particular probe or probe set. In the case of a probe set, constituent probes of the probe set can be combined prior to placement on the solid support or by serial placement of constituent probes at a same position on the solid support.

A microarray can be assembled using any suitable method known to one of skill in the art, and any one microarray configuration or method of construction is not considered to be a limitation of the present invention. Representative microarray formats that can be used in accordance with the methods of the present invention are described herein below.

V.A. Array Substrate and Configuration

The substrate for printing the array should be substantially rigid and amenable to DNA immobilization and detection methods (e.g., in the case of fluorescent detection, the substrate must have low background fluorescence in the region of the fluorescent dye excitation wavelengths). The substrate can be nonporous or porous as determined most suitable for a particular application. Representative substrates include but are not limited to a glass microscope slide, a glass coverslip, silicon, plastic, a polymer matrix, an agar gel, a polyacrylamide gel, and a membrane, such as a nylon, nitrocellulose or ANAPORE™ (Whatman of Maidstone, United Kingdom) membrane.

Porous substrates (membranes and polymer matrices) are preferred in that they permit immobilization of relatively large amount of probe molecules and provide a three-dimensional hydrophilic environment for biomolecular interactions to occur (Dubiley et al. (1997) Nuc Acids Res 25:2259-2265; Yershov et al. (1996) Proc Natl Acad Sci USA 93:4319-4918). A BIOCHIP ARRAYER™ dispenser (Packard Instrument Company of Meriden, Conn., United States of America) can effectively dispense probes onto membranes such that the spot size is consistent among spots whether one, two, or four droplets were dispensed per spot (Englert (2000) in Schena, ed., Microarray Biochip Technology, pp. 231-246, Eaton Publishing, Natick, Mass., United States of America).

A microarray substrate for use in accordance with the methods of the present invention can have either a two-dimensional (planar) or a three-dimensional (non-planar) configuration. An exemplary three-dimensional microarray is the FLOW-THRU™ chip (Gene Logic, Inc. of Gaithersburg, Md., United States of America), which has implemented a gel pad to create a third dimension. Such a three-dimensional microarray can be constructed of any suitable substrate, including glass capillary, silicon, metal oxide filters, or porous polymers. See Yang et al. (1998) Science 282:2244-2246 and Steel et al. (2000) in Schena, ed., Microarray Biochip Technology, pp. 87-118, Eaton Publishing, Natick, Mass., United States of America.

Briefly, a FLOW-THRU™ chip (Gene Logic, Inc.) comprises a uniformly porous substrate having pores or microchannels connecting upper and lower faces of the chip. Probes are immobilized on the walls of the microchannels and a hybridization solution comprising sample nucleic acids can flow through the microchannels. This configuration increases the capacity for probe and target binding by providing additional surface relative to two-dimensional arrays. See U.S. Pat. No. 5,843,767.

V.B. Surface Chemistry

The particular surface chemistry employed is inherent in the microarray substrate and substrate preparation. Probe immobilization of nucleic acids probes post-synthesis can be accomplished by various approaches, including adsorption, entrapment, and covalent attachment. Preferably, the binding technique does not disrupt the activity of the probe.

For substantially permanent immobilization, covalent attachment is preferred. Since few organic functional groups react with an activated silica surface, an intermediate layer is advisable for substantially permanent probe immobilization. Functionalized organosilanes can be used as such an intermediate layer on glass and silicon substrates (Liu & Hlady (1996) Coll Sur B 8:25-37; Shriver-Lake (1998) in Cass & Ligler, eds., Immobilized Biomolecules in Analysis, pp.1-14, Oxford Press, Oxford, United Kingdom). A hetero-bifunctional cross-linker requires that the probe have a different chemistry than the surface, and is preferred to avoid linking reactive groups of the same type. A representative hetero-bifunctional cross-linker comprises gamma-maleimidobutyryloxy-succimide (GMBS) that can bind maleimide to a primary amine of a probe. Procedures for using such linkers are known to one of skill in the art and are summarized by Hermanson (1990) Bioconjugate Techniques, Academic Press, San Diego, Calif. A representative protocol for covalent attachment of DNA to silicon wafers is described by O'Donnell et al. (1997) Anal Chem 69:2438-2443.

When using a glass substrate, the glass should be substantially free of debris and other deposits and have a substantially uniform coating. Pretreatment of slides to remove organic compounds that can be deposited during their manufacture can be accomplished, for example, by washing in hot nitric acid. Cleaned slides can then be coated with 3-aminopropyltrimethoxysilane using vapor-phase techniques. After silane deposition, slides are washed with deionized water to remove any silane that is not attached to the glass and to catalyze unreacted methoxy groups to cross-link to neighboring silane moieties on the slide. The uniformity of the coating can be assessed by known methods, for example electron spectroscopy for chemical analysis (ESCA) or ellipsometry (Ratner & Castner (1997) in Vickerman, ed., Surface Analysis: The Principal Techniques, John Wiley & Sons, New York; Schena et al. (1995) Science 270:467-470). See also Worley et al. (2000) in Schena, ed., Microarray Biochip Technology, pp. 65-86, Eaton Publishing, Natick, Mass., United States of America.

For attachment of probes greater than about 300 base pairs, noncovalent binding is suitable. A representative technique for noncovalent linkage involves use of sodium isothiocyanate (NaSCN) in the spotting solution, as described in Example 7. When using this method, amino-silanized slides are preferred in that this coating improves nucleic acid binding when compared to bare glass. This method works well for spotting applications that use about 100 ng/μl (Worley et al. (2000) in Schena, ed., Microarray Biochip Technology, pp. 65-86, Eaton Publishing, Natick, Mass., United States of America).

In the case of nitrocellulose or nylon membranes, the chemistry of nucleic acid binding chemistry to these membranes has been well characterized (Southern (1975) J Mol Biol 98:503-517); Maniatis et al. (1989) Molecular Cloning: A Laboratory Manual, Cold Spring Harbor Press, Cold Spring Harbor, N.Y.).

V.C. Arraying Technigues

A microarray for the detection of pathogens in a biological sample can be constructed using any one of several methods available in the art, including but not limited to photolithographic and microfluidic methods, further described herein below. Preferably, the method of construction is flexible, such that a microarray can be tailored for a particular purpose.

As is standard in the art, a technique for making a microarray should create consistent and reproducible spots. Each spot is preferably uniform, and appropriately spaced away from other spots within the configuration. A solid support for use in the present invention preferably comprises about 10 or more spots, or more preferably about 100 or more spots, even more preferably about 1,000 or more spots, and still more preferably about 10,000 or more spots. Also preferably, the volume deposited per spot is about 10 picoliters to about 10 nanoliters, and more preferably about 50 picoliters to about 500 picoliters. The diameter of a spot is preferably about 50 μm to about 1000 μm, and more preferably about 100 μm to about 250 μm.

Light-Directed Synthesis. This technique was developed by Fodor et al. (Fodor et al. (1991) Science 251:767-773; Fodor et al. (1993) Nature 364:555-556; U.S. Pat. No. 5,445,934), and commercialized by Affymetrix of Santa Clara, Calif., United States of America. Briefly, the technique uses precision photolithographic masks to define the positions at which single, specific nucleotides are added to growing single-stranded nucleic acid chains. Through a stepwise series of defined nucleotide additions and light-directed chemical linking steps, high-density arrays of defined oligonucleotides are synthesized on a solid substrate. A variation of the method, called Digital Optical Chemistry, employs mirrors to direct light synthesis in place of photolithographic masks (International Publication No. WO 99/63385). This approach is generally limited to probes of about 25 nucleotides in length or less. See also Warrington et al. (2000) in Shena, ed., Microarray Biochip Technology, pp. 119-148, Eaton Publishing, Natick, Mass., United States of America.

Contact Printing. Several procedures and tools have been developed for printing microarrays using rigid pin tools. In surface contact printing, the pin tools are dipped into a sample solution, resulting in the transfer of a small volume of fluid onto the tip of the pins. Touching the pins or pin samples onto a microarray surface leaves a spot, the diameter of which is determined by the surface energies of the pin, fluid, and microarray surface. Typically, the transferred fluid comprises a volume in the nanoliter or picoliter range.

One common contact printing technique uses a solid pin replicator. A replicator pin is a tool for picking up a sample from one stationary location and transporting it to a defined location on a solid support. A typical configuration for a replicating head is an array of solid pins, generally in an 8×12 format, spaced at 9-mm centers that are compatible with 96- and 384-well plates. The pins are dipped into the wells, lifted, moved to a position over the microarray substrate, lowered to touch the solid support, whereby the sample is transferred. The process is repeated to complete transfer of all the samples. See Maier et al. (1994) J Biotechnol 35:191-203. A recent modification of solid pins involves the use of solid pin tips having concave bottoms, which print more efficiently than flat pins in some circumstances. See Rose (2000) in Shena, ed., Microarray Biochip Technology, pp. 19-38, Eaton Publishing, Natick, Mass., United States of America.

Solid pins for microarray printing can be purchased, for example, from TeleChem International, Inc. of Sunnyvale, Calif. in a wide range of tip dimensions. The CHIPMAKER™ and STEALTH™ pins from TeleChem contain a stainless steel shaft with a fine point. A narrow gap is machined into the point to serve as a reservoir for sample loading and spotting. The pins have a loading volume of 0.2 μl to 0.6 μl to create spot sizes ranging from 75 μm to 360 μm in diameter.

To permit the printing of multiple arrays with a single sample loading, quill-based array tools, including printing capillaries, tweezers, and split pins have been developed. These printing tools hold larger sample volumes than solid pins and therefore allow the printing of multiple arrays following a single sample loading. Quill-based arrayers withdraw a small volume of fluid into a depositing device from a microwell plate by capillary action. See Schena et al. (1995) Science 270:467-470. The diameter of the capillary typically ranges from about 10 μm to about 100 μm. A robot then moves the head with quills to the desired location for dispensing. The quill carries the sample to all spotting locations, where a fraction of the sample is deposited. The forces acting on the fluid held in the quill must be overcome for the fluid to be released. Accelerating and then decelerating by impacting the quill on a microarray substrate accomplishes fluid release. When the tip of the quill hits the solid support, the meniscus is extended beyond the tip and transferred onto the substrate. Carrying a large volume of sample fluid minimizes spotting variability between arrays. Because tapping on the surface is required for fluid transfer, a relatively rigid support, for example a glass slide, is appropriate for this method of sample delivery.

A variation of the pin printing process is the PIN-AND-RING™ technique developed by Genetic MicroSystems Inc. of Woburn, Mass., United States of America. This technique involves dipping a small ring into the sample well and removing it to capture liquid in the ring. A solid pin is then pushed through the sample in the ring, and the sample trapped on the flat end of the pin is deposited onto the surface. See Mace et al. (2000) in Shena, ed., Microarray Biochip Technology, pp. 39-64, Eaton Publishing, Natick, Mass., United States of America. The PIN-AND-RING™ technique is suitable for spotting onto rigid supports or soft substrates such as agar, gels, nitrocellulose, and nylon. A representative instrument that employs the PIN-AND-RING™ technique is the 417™ Arrayer available from Affymetrix of Santa Clara, Calif., United States of America.

Additional procedural considerations relevant to contact printing methods, including array layout options, print area, print head configurations, sample loading, preprinting, microarray surface properties, sample solution properties, pin velocity, pin washing, printing time, reproducibility, and printing throughput are known in the art, and are summarized by Rose (2000) in Shena, ed., Microarray Biochip Technology, pp. 19-38, Eaton Publishing, Natick, Mass., United States of America.

Noncontact Ink-Jet Printing. A representative method for noncontact ink-jet printing uses a piezoelectric crystal closely apposed to the fluid reservoir. One configuration places the piezoelectric crystal in contact with a glass capillary that holds the sample fluid. The sample is drawn up into the reservoir and the crystal is biased with a voltage, which causes the crystal to deform, squeeze the capillary, and eject a small amount of fluid from the tip. Piezoelectric pumps offer the capability of controllable, fast jetting rates and consistent volume deposition. Most piezoelectric pumps are unidirectional pumps that need to be directly connected, for example by flexible capillary tubing, to a source of sample supply or wash solution. The capillary and jet orifices should be of sufficient inner diameter so that molecules are not sheared. The void volume of fluid contained in the capillary typically ranges from about 100 μl to about 500 μl and generally is not recoverable. See U.S. Pat. No. 5,965,352.

Devices that provide thermal pressure, sonic pressure, or oscillatory pressure on a liquid stream or surface can also be used for ink-jet printing. See Theriault et al. (1999) in Schena, ed., DNA Microarrays: A Practical Approach, pp.101-120, Oxford University Press Inc., New York, N.Y.

Syringe-Solenoid Printing. Syringe-solenoid technology combines a syringe pump with a microsolenoid valve to provide quantitative dispensing of nanoliter sample volumes. A high-resolution syringe pump is connected to both a high-speed microsolenoid valve and a reservoir through a switching valve. For printing microarrays, the system is filled with a system fluid, typically water, and the syringe is connected to the microsolenoid valve. Withdrawing the syringe causes the sample to move upward into the tip. The syringe then pressurizes the system such that opening the microsolenoid valve causes droplets to be ejected onto the surface. With this configuration, a minimum dispense volume is on the order of 4 nl to 8 nl. The positive displacement nature of the dispensing mechanism creates a substantially reliable system. See U.S. Pat. Nos. 5,743,960 and 5,916,524.

Electronic Addressing. This method involves placing charged molecules at specific positions on a blank microarray substrate, for example a NANOCHIP™ substrate (Nanogen Inc. of San Diego, Calif.). A nucleic acid probe is introduced to the microchip, and the negatively-charged probe moves to the selected charged position, where it is concentrated and bound. Serial application of different probes can be performed to assemble an array of probes at distinct positions. See U.S. Pat. No. 6,225,059 and International Publication No. WO 01/23082.

Nanoelectrode Synthesis. An alternative array that can also be used in accordance with the methods of the present invention provides ultra small structures (nanostructures) of a single or a few atomic layers synthesized on a semiconductor surface such as silicon. The nanostructures can be designed to correspond precisely to the three-dimensional shape and electrochemical properties of molecules, and thus can be used to recognize nucleic acids of a particular nucleotide sequence. See U.S. Pat. No. 6,123,819.

VI. Hybridization

VI.A. General Considerations

The terms “specifically hybridizes” and “selectively hybridizes” each refer to binding, duplexing, or hybridizing of a molecule only to a particular nucleotide sequence under stringent conditions when that sequence is present in a complex nucleic acid mixture (e.g., total cellular DNA or RNA).

The phrase “substantially hybridizes” refers to complementary hybridization between a probe nucleic acid molecule and a substantially identical target nucleic acid molecule as defined herein. Substantial hybridization is generally permitted by reducing the stringency of the hybridization conditions using art-recognized techniques.

“Stringent hybridization conditions” and “stringent hybridization wash conditions” in the context of nucleic acid hybridization experiments are both sequence- and environment-dependent. Longer sequences hybridize specifically at higher temperatures. Generally, highly stringent hybridization and wash conditions are selected to be about 5° C. lower than the thermal melting point (Tₘ) for the specific sequence at a defined ionic strength and pH. The Tₘ is the temperature (under defined ionic strength and pH) at which 50% of the target sequence hybridizes to a perfectly matched probe. Very stringent conditions are selected to be equal to the Tₘ for a particular probe. Typically, under “stringent conditions” a probe hybridizes specifically to its target sequence, but to no other sequences.

An extensive guide to the hybridization of nucleic acids is found in Tijssen (1993) Laboratory Techniques in Biochemistry and Molecular Biology-Hybridization with Nucleic Acid Probes, part I chapter 2, Elsevier, New York, N.Y. In general, a signal to noise ratio of 2-fold (or higher) than that observed for a negative control probe in a same hybridization assay indicates detection of specific or substantial hybridization.

VI.B. Hybridization on a Solid Support

In another embodiment of the invention, an amplified and labeled nucleic acid sample is hybridized to pathogen-specific probes or probe sets that are immobilized on a continuous solid support comprising a plurality of identifying positions. Representative formats of such solid supports are described herein above under the heading Microarray Formats.

Representative hybridization conditions are set forth in Example 8. For some high-density glass-based microarray experiments, hybridization at 65° C. is too stringent for typical use, at least in part because the presence of fluorescent labels destabilizes the nucleic acid duplexes (Randolph & Waggoner (1997) Nuc Acids Res 25:2923-2929). Alternatively, hybridization can be performed in a formamide-based hybridization buffer as described in Piétu et al. (1996) Genome Res 6:492-503.

A microarray format can be selected for use based on its suitability for electrochemical-enhanced hybridization. Provision of an electric current to the microarray, or to one or more discrete positions on the microarray facilitates localization of a target nucleic acid sample near probes immobilized on the microarray surface. Concentration of target nucleic acid near arrayed probe accelerates hybridization of a nucleic acid of the sample to a probe. Further, electronic stringency control allows the removal of unbound and nonspecifically bound DNA after hybridization. See U.S. Pat. Nos. 6,017,696 and 6,245,508.

VI.C. Hybridization in Solution

In another embodiment of the invention, an amplified and labeled nucleic acid sample is hybridized to one or more pathogen-specific probes in solution. Representative stringent hybridization conditions for complementary nucleic acids having more than about 100 complementary residues are overnight hybridization in 50% formamide with 1 mg of heparin at 42° C. An example of highly stringent wash conditions is 15 minutes in 0.1×SSC, 5M NaCl at 65° C. An example of stringent wash conditions is 15 minutes in 0.2×SSC buffer at 65° C. (See Sambrook et al., eds. (1989) Molecular Cloning: A Laboratory Manual, Cold Spring Harbor Laboratory Press, Cold Spring Harbor, N.Y. for a description of SSC buffer). A high stringency wash can be preceded by a low stringency wash to remove background probe signal. An example of medium stringency wash conditions for a duplex of more than about 100 nucleotides, is 15 minutes in 1×SSC at 45° C. An example of low stringency wash for a duplex of more than about 100 nucleotides, is 15 minutes in 4-6×SSC at 40° C. Stringent conditions can also be achieved with the addition of destabilizing agents such as formamide.

For short probes (e.g., about 10 to 50 nucleotides), stringent conditions typically involve salt concentrations of less than about 1M Na⁺ ion, typically about 0.01M to 1M Na⁺ ion concentration (or other salts) at pH 7.0-8.3, and the temperature is typically at least about 30° C.

Optionally, nucleic acid duplexes or hybrids can be captured from the solution for subsequent analysis, including detection assays. For example, in a simple assay, a single pathogen-specific probe set is hybridized to an amplified and labeled RNA sample derived from a target nucleic acid sample. Following hybridization, an antibody that recognizes DNA:RNA hybrids is used to precipitate the hybrids for subsequent analysis. The presence of the pathogen is determined by detection of the label in the precipitate.

Alternate capture techniques can be used as will be understood to one of skill in the art, for example, purification by a metal affinity column when using pathogen-specific probes comprising a histidine tag. As another example, the hybridized sample can be hydrolyzed by alkaline treatment wherein the double-stranded hybrids are protected while non-hybridizing single-stranded template and excess probe are hydrolyzed. The hybrids are then collected using any nucleic acid purification technique for further analysis.

To assess the presence of multiple pathogens simultaneously, probes or probe sets can be distinguished by differential labeling of pathogen-specific probes or pathogen-specific probe sets. Alternatively, probes or probe sets can be spatially separated in different hybridization vessels. Representative embodiments of each approach are described herein below.

In one embodiment, a probe or probe set having a unique label is prepared for each pathogen to be detected. For example, a first probe or probe set can be labeled with a first fluorescent label, and a second probe or probe set can be labeled with a second fluorescent label. Multi-labeling experiments should consider label characteristics and detection techniques to optimize detection of each label. Representative first and second fluorescent labels are Cy3 and Cy5 (Amersham Pharmacia Biotech of Piscataway, N.J., United States of America), which can be analyzed with good contrast and minimal signal leakage.

A unique label for each probe or probe set can further comprise a labeled microsphere to which a probe or probe set is attached. A representative system is LabMAP (Luminex Corporation of Austin, Tex., United States of America). Briefly, LabMAP (Laboratory Multiple Analyte Profiling) technology involves performing molecular reactions, including hybridization reactions, on the surface of color-coded microscopic beads called microspheres. When used in accordance with the methods of the present invention, an individual pathogen-specific probe or probe set is attached to beads having a single color-code such that they can be identified throughout the assay. Successful hybridization is measured using a detectable label of the amplified nucleic acid sample, wherein the detectable label can be distinguished from each color-code used to identify individual microspheres. Following hybridization of the randomly amplified, labeled nucleic acid sample with a set of microspheres comprising pathogen-specific probe sets, the hybridization mixture is analyzed to detect the signal of the color-code as well as the label of a sample nucleic acid bound to the microsphere. See Vignali (2000) J Immunol Methods 243(1-2):243-255, Smith et al. (1998) Clin Chem 44(9):2054-2056, and International Publication Nos. WO 01/13120, WO 01/14589, WO 99/19515, and WO 97/14028.

In another embodiment, multiple pathogens can be detected simultaneously by distribution of each of a plurality of pathogen-specific probes or probe sets to single wells of a multi-well plate, such that the position of the well defines each probe or probe set. In the case of a probe set, constituent probes of the probe set can be combined prior to placement in a well or by serial placement of constituent probes in a single well. Optionally, probes or probe sets are immobilized in the well. Amplified nucleic acids of a biological sample and hybridization solution are provided to each of the wells of the plate, and successful hybridization is detected by any suitable method.

VII. Detection

Methods for detecting a hybridization duplex or triplex are selected according to the label employed.

In the case of a radioactive label (e.g., ³²P-dNTP) detection can be accomplished by autoradiography or by using a phosphorimager as is known to one of skill in the art. Preferably, a detection method can be automated and is adapted for simultaneous detection of numerous samples.

Common research equipment has been developed to perform high-throughput fluorescence detecting, including instruments from GSI Lumonics (Watertown, Mass., United States of America), Amersham Pharmacia Biotech/Molecular Dynamics (Sunnyvale, Calif., United States of America), Applied Precision Inc. (Issauah, Wash., United States of America), Genomic Solutions Inc. (Ann Arbor, Mich., United States of America), Genetic MicroSystems Inc. (Woburn, Mass., United States of America), Axon (Foster City, Calif., United States of America), Hewlett Packard (Palo Alto, Calif., United States of America), and Virtek (Woburn, Mass., United States of America). Most of the commercial systems use some form of scanning technology with photomultiplier tube detection. Criteria for consideration when analyzing fluorescent samples are summarized by Alexay et al. (1996) The International Society of Optical Engineering 2705/63.

In another embodiment, a nucleic acid sample or pathogen-specific probes are labeled with far infrared, near infrared, or infrared fluorescent dyes. Following hybridization, the mixture of randomly amplified nucleic acids and pathogen-specific probes is scanned photoelectrically with a laser diode and a sensor, wherein the laser scans with scanning light at a wavelength within the absorbance spectrum of the fluorescent label, and light is sensed at the emission wavelength of the label. See U.S. Pat. Nos. 6,086,737; 5,571,388; 5,346,603; 5,534,125; 5,360,523; 5,230,781; 5,207,880; and 4,729,947. An ODYSSEY™ infrared imaging system (Li-Cor, Inc. of Lincoln, Nebr., United States of America) can be used for data collection and analysis.

If an epitope label has been used, a protein or compound that binds the epitope can be used to detect the epitope. For example, an enzyme-linked protein can be subsequently detected by development of a calorimetric or luminescent reaction product that is measurable using a spectrophotometer or luminometer, respectively.

In one embodiment, INVADER® technology (Third Wave Technologies of Madison, Wis., United States of America) is used to detect target nucleic acid/probe complexes. Briefly, a nucleic acid cleavage site (such as that recognized by a variety of enzymes having 5′ nuclease activity) is created on a target sequence, and the target sequence is cleaved in a site-specific manner, thereby indicating the presence of specific nucleic acid sequences or specific variations thereof. See U.S. Pat. Nos. 5,846,717; 5,985,557; 5,994,069; 6,001,567; and 6,090,543.

In another embodiment, target nucleic acid/probe complexes are detected using an amplifying molecule, for example a poly-dA oligonucleotide as described by Lisle et al. (2001) BioTechniques 30(6):1268-1272. Briefly, a tethered probe is employed against a target nucleic acid having a complementary nucleotide sequence. A target nucleic acid having a poly-dT sequence, which can be added to any nucleic acid sequence using methods known to one of skill in the art, hybridizes with an amplifying molecule comprising a poly-dA oligonucleotide. Short oligo-dT₄₀ signaling moieties are labeled with any suitable label (e.g., fluorescent, chemiluminescent, radioisotopic labels). The short oligo-dT₄₀ signaling moieties are subsequently hybridized along the molecule, and the label is detected.

The present invention also envisions use of electrochemical technology for detecting a nucleic acid hybrid according to the disclosed method. In this case, the detection method relies on the inherent properties of DNA, and thus a detectable label on the target sample or the probe/probe set is not required. Preferably, probe-coupled electrodes are multiplexed to simultaneously detect multiple pathogens using any suitable microarray or multiplexed liquid hybridization format. To enable detection, pathogen-specific and control probes are synthesized with substitution of the non-physiological nucleic acid base inosine for guanine, and subsequently coupled to an electrode. Following hybridization of a nucleic acid sample with probe-coupled electrodes, a soluble redox-active mediator (e.g., ruthenium 2,2′-bipyridine) is added, and a potential is applied to the sample. In the absence of guanine, each mediator is oxidized only once. However, when a guanine-containing nucleic acid is present, by virtue of hybridization of a sample nucleic acid molecule to the probe, a catalytic cycle is created that results in the oxidation of guanine and a measurable current enhancement. See U.S. Pat. Nos. 6,127,127; 5,968,745; and 5,871,918.

Surface plasmon resonance spectroscopy can also be used to detect hybridization duplexes formed between a randomly amplified nucleic acid and a pathogen-specific probe as disclosed herein. See e.g., Heaton et al. (2001) Proc Natl Acad Sci USA 98(7):3701-3704; Nelson et al. (2001) Anal Chem 73(1):1-7; and Guedon et al. (2000) Anal Chem 72(24):6003-6009.

VIII. Data Analysis

The present invention provides methods for detecting a pathogen that rely on absolute detection. For example, an initial diagnostic survey can comprise determining the presence of a pathogen at any detectable level. Preferably, a level of hybridization to a pathogen-specific probe is greater than about two-fold higher than a level of detection of a negative control probe, more preferably greater than about five-fold higher than a level of detection of a negative control probe, and still more preferably greater than about ten-fold higher than a level of detection of a negative control probe.

Hybridization data can be further evaluated to assess overall quality and reproducibility of the assay. For this purpose, relevant measures include: (i) the average variation of hybridization replicates (defined as the standard deviation of hybridization replicate values/mean, and (ii) the R² value for the least squares line drawn through the scatter plot of hybridization replicates. For a glass slide microarray, a typical average variation is less than about 22%.

Preferably, data analysis also comprises characterization of hybridization performance features displayed by probes used in accordance with the disclosed methods. To facilitate probe selection, the results of such analysis can be stored as a database or otherwise saved for future reference. For example, a probe can be selected for a particular application based on its generation of minimal background in hybridization assays.

Numerous software packages have been developed for microarray data analysis, and an appropriate program can be selected according to the array format and detection method. Some products, including ARRAYGAUGE™ software (Fujifilm Medical Systems Inc. of Stamford, Conn., United States of America) and IMAGEMASTER ARRAY 2™ software (Amersham Pharmacia Biotech of Piscataway, N.J., United States of America), accept images from most microarray scanners and offer substantial flexibility for analyzing data generated by different instruments and array types. Other microarray analysis software products are designed specifically for use with particular array scanners or for particular array formats. A survey of currently available microarray analysis software packages can be found in Brush (2001) The Scientist 15(9);25-28. In addition, the guidance presented herein provides for the development of software and/or databases by one of ordinary skill in the art, to facilitate analysis of data obtained by performing the method of the present invention.

IX. Applications

The methods of the present invention have broad utility for detecting pathogens in a variety of biological samples, including samples suspected of containing a pathogen associated with biological warfare or bioterrorism.

The methods can be modified for simultaneous detection of a multitude of infectious agents, minimizing a reliance on a presupposed presence of an infectious agent and facilitating determination of conditions resulting from the presence of multiple infectious agents. Preferably, a hybridization assay of the present invention employs one or more probes representing every known pathogen for a particular host species or for a particular biological sample.

The disclosed methods provide detection of unculturable infectious agents, facilitate more rapid identification of slow growing or fastidious agents, and minimize laboratory risk of infection associated with culturing methods. The present invention also provides methods for detecting nonviable pathogenic agents that can be present after the initiation of antimicrobial or antiviral therapies, during a latent infection, or in an extant sample.

In summary, the present invention provides a method for detecting a pathogen in a biological sample that offers a potential for improved sensitivity and efficiency of detection, even in cases wherein a pathogen is not suspected to be present in the sample. The disclosed detection methods can also be used for genotyping variant forms of a pathogen, for example to distinguish between drug-resistant and drug-susceptible forms. In cases of a suspected pathogen or group of pathogens, probes can be variably selected to detect the suspected pathogens (e.g., pathogens that infect the respiratory tract, pathogens that frequently infect children). The method thus facilitates early and accurate detection of an infectious agent and subsequent monitoring and management of such a presence.

### EXAMPLES

The following Examples have been included to illustrate modes of the invention. Certain aspects of the following Examples are described in terms of techniques and procedures found or contemplated by the present co-inventors to work well in the practice of the invention. These Examples illustrate standard laboratory practices of the co-inventors. In light of the present disclosure and the general level of skill in the art, those of skill can appreciate that the following Examples are intended to be exemplary only and that numerous changes, modifications, and alterations can be employed without departing from the scope of the invention.

### Example 1

Isolation of RNA from a Clinical Sample

To a clinical sample on ice (approximately equivalent to about 1×10⁶-1×10⁷ cells), 500 μl of guanidinium isothiocyantate stock solution (4M guanidinium isothiocyanate, 25 mM sodium citrate pH 7.0, 0.5% sarcosyl, 0.1M 2-mercaptoethanol) is added. The sample is homogenized and briefly centrifuged to remove insoluble material. 50 μl of 3M sodium acetate pH 4.0 is added to the supernatant, and the mixture is agitated to facilitate mixing followed by brief centrifugation. 600 μl of phenol:chloroform:isoamyl alcohol (25:24:1, vol/vol) saturated with 10 mM Tris-HCl pH 7.5 and 1 mM EDTA is added to the supernatant, and the mixture is agitated to facilitate mixing followed by incubation on ice for 15 minutes. The mixture is centrifuged at 10,000×g for 20 minutes at 4° C., and the aqueous layer is transferred to a new tube. To the aqueous layer, 1 ml of ice-cold absolute ethanol or 500 μl of isopropanol is added. The sample is mixed gently and the RNA is allowed to precipitate overnight at −20° C. The sample is centrifuged at 10,000×g for 20 minutes at 4° C. to pellet the RNA. The RNA is resuspended in 300-400 μl of guanidinium isothiocyanate stock solution, and 2 volumes of ice-cold ethanol or an equal volume of isopropanol is added. The RNA is again allowed to precipitate overnight at −20° C. The RNA is pelleted by centrifugation at 10,000×g for 20 minutes at 4° C., rinsed in ice-cold 75% ethanol, and briefly dried. For short-term storage or immediate use, the pellet is resuspended in 50 μl of RNAase-free water and stored at 4° C. For long-term storage, the pellet can be stored in 75% ethanol at 4° C.

### Example 2

Random Hexamer Primer PCR Amplification of Genomic DNA

High molecular weight DNA is extracted from a biological sample using a standard method. The high molecular weight DNA is digested in an appropriate volume of 200 μg/ml proteinase K, 10 mM Tris-HCl (pH 8.3), 50 mM KCl, and 0.1% TRITON-X-100® detergent (available from Sigma Chemical Company of St. Louis, Mo., United States of America) for about 3 hours or up to 3 days at 37° C. Digested DNA can be purified prior to amplification, for example by precipitation with alcohol.

RP-PCR is performed in two phases. Phase I reactions are run in a 10 μl solution containing 0.025 units of AMPLITAQ® DNA polymerase (PerkinElmer of Wellesley, Mass., United States of America), 10 mM Tris (pH 8.3), 50 mM KCl, 1 mM MgCl₂, 0.001% gelatin, 0.02 mM each dNTP, 10 μM of random hexamers (Boehringer of Germany), and 4 μl of a digested DNA sample. The DNA sample can comprise 4 pg to 40 ng of high molecular weight DNA. The DNA is denatured at 95° C. for 5 minutes, followed by 10 cycles in a thermal cycler (Hybaid of United Kingdom): 1 minute at 95° C., 1 minute at 37° C., and 5 minutes at 50° C. To each reaction, a 40 μl solution containing 0.5 units AMPLITAQ® enzyme, 1.5 mM MgCl₂, 0.2 mM of each dNTP, 10 mM Tris (pH 8.3), 50 mM KCl, 1 mM MgCl₂, and 0.001% gelatin was added. Each reaction is subjected to 40 additional cycles of 1 minute at 95° C., 1 minute at 55° C., and 2 minutes at 72° C.

### Example 3

Random Amplification of Genomic DNA Using a RAPD-Type Primer

High molecular weight DNA is extracted from a biological sample using a standard method. The high molecular weight DNA is digested in an appropriate volume of 200 μg/ml proteinase K, 10 mM Tris-HCl (pH 8.3), 50 mM KCl, 0.1% TRITON-X-100® detergent (available from Sigma Chemical Company of St. Louis, Mo., United States of America) for about 3 hours or up to 3 days at 37° C. Digested DNA can be purified prior to amplification, for example by precipitation with alcohol.

A RAPD-type primer comprising a 10-nucleotide arbitrary sequence is obtained from Operon Technologies of Alameda, Calif., United States of America. Random amplification using the RAPD-type primer is performed using genomic DNA from a biological sample as a template as described by Hopkins & Hilton (2001) BioTechniques 30(6):1262-1267.

### Example 4

Random Amplification of RNA (aRNA)

Total RNA is isolated from the biological sample according to standard methods. Approximately 40 μg of total RNA is primed with 100 ng of primer comprising a poly(dT) stretch and a sequence comprising a T7 RNA polymerase binding site (representative embodiment set forth as SEQ ID NO: 1; aaacgacggccagtgaattgtaatacgactcactataggcgcttttttttttttttt). The RNA/primer mixture is denatured by heating at 95° C. for 3 minutes. First strand cDNA synthesis is performed using avian myeloblastoma virus reverse transcriptase.

First strand cDNA is treated with S1 nuclease, end repaired, and ethanol precipitated. The cDNA pellet is dissolved in 10 μl of TE and drop-dialyzed for 4 hours against 50 ml of TE using a 0.025 mm nitrocellulose filter (Millipore Corporation of Bedford, Mass., United States of America).

For amplification of RNA, a 20-μl reaction mixture is prepared containing 3 ng of cDNA, 40 mM Tris (pH 7.5), 6 mM MgCl₂, 10 mM NaCl, 2 mM spermidine, 10 mM DTT, 500 μM each ATP, GTP, UTP, and CTP, 10 units RNasin, and 80 units T7 RNA polymerase. The reaction is carried out for 2 hours at 37° C. Optionally, a radiolabeled nucleotide, or other labeled nucleotide analog, can be included to determine the amplification efficiency. For example, 12.5 μM CTP and 30 μCi ³²P-CTP can replace 500 μM CTP in the above procedure. The RNA is then extracted with phenol/chloroform and precipitated with ethanol. At this point, the RNA can be labeled according to methods known in the art, and subsequently used as probe.

Optionally, the RNA can be reamplified by dissolving the pellet in 20 μl of H₂O, adding 10-100 ng of random hexanucleotide primers, 3 μl of 10×RT buffer (500 mM Tris, pH 8.3, 1.2 M KCl, 100 mM MgCl₂, 0.5 mM NaPPᵢ), 3 μl of 100 mM dithiotheitol (DTT), dNTPs to 250 μM, H₂O to 27.5 μl, 0.5 μl RNasin, and 2 μl reverse transcriptase, and incubating the reaction mixture at 37° C. for 1 hour. The mixture is then extracted with phenol/chloroform and precipitated with ethanol. The pellet is dissolved in 10 μl H₂O, heat denatured at 95° C. for 2 minutes, and quick-cooled on ice. Second-strand cDNA is synthesized by adding 100 ng of oligo(dT)-T7 amplification oligonucleotide (e.g., SEQ ID NO: 1), 2 μl of 10×KFI buffer (200 mM Tris, pH 7.5, 100 mM MgCl₂, 50 mM DTT, 50 mM NaCl), dNTPs to 250 μM, H₂O to 17 μl, 1 μl T4 DNA polymerase, and 1 μl Klenow. The reaction mixture is incubated at 14° C. for 2 hours, extracted with phenol/chloroform, and precipitated with ethanol. The pellet is dissolved in 20 μl of TE and drop-dialyzed. RNA can then be synthesized as described above.

### Example 5

Preparation of Fungal Probes Using a RAPD-Type Primer

Isolated genomic DNA is prepared from Aspergillus flavus, Fusarium verticilliodes, Aspergillus niger, and Cercospora kikuchii according to methods disclosed herein and known in the art. A RAPD-type primer comprising a 10-nucleotide arbitrary sequence is obtained from Operon Technologies of Alameda, Calif., United States of America. Random amplification using the RAPD-type primer is performed using pure fungal genomic DNA as a template as described by Hopkins & Hilton (2001) BioTechniques 30(6):1262-1267. Amplification products are resolved using gel electrophoresis. Amplicons that are uniquely amplified from each fungus are purified from the gel and sequenced to confirm correct amplification of the desired sequence, as compared to a reference sequence. Alternatively, the amplicons can be hybridized to a reference sequence under hybridization conditions of suitable stringency as described herein and as would be apparent to one of ordinary skill in the art after a review of the disclosure of the present invention presented herein.

Preferably, pathogen-specific probes that are prepared using a RAPD-type primer are hybridized to a nucleic acid sample that has been amplified using a same RAPD-type primer, as described in Example 3.

### Example 6

Fluorescent Labeling of Nucleic Acids

A nucleic acid sample can be used as a template for direct incorporation of fluorescent nucleotide analogs (e.g., Cy3-dUTP and Cy5-dUTP, available from Amersham Pharmacia Biotech of Piscataway, N.J., United States of America) by a randomly primed polymerization reaction. In brief, a 50 μl labeling reaction can contain 2 μg of template DNA, 5 μl of 10×buffer, 1.5 μl of fluorescent dUTP, 0.5 μl each of dATP, dCTP, and dGTP, 1 μl of random hexamers and decamers, and 2 μl of Klenow (E. coli DNA polymerase 3′ to 5′ exo—from New England Biolabs of Beverly, Mass., United States of America).

### Example 7

Noncovalent Binding of Nucleic Acid Probes onto Glass

PCR fragments are suspended in a solution of 3 to 5M NaSCN and spotted onto amino-silanized slides using a GMS ₄₁₇™ arrayer from Affymetrix of Santa Clara, Calif., United States of America. After spotting, the slides are heated at 80° C. for 2 hours to dehydrate the spots. Prior to hybridization, the slides are washed in isopropanol for 10 minutes, followed by washing in boiling water for 5 minutes. The washing steps remove any nucleic acid that is not bound tightly to the glass and help to reduce background created by redistribution of loosely attached DNA during hybridization. Contaminants such as detergents and carbohydrates should be minimized in the spotting solution. See also Maitra & Thakur (1994) Indian J Biochem Biophys 31:97-99; and Maitra & Thakur (1992) Curr Sci 62:586-588.

### Example 8

Hybridization of Target Nucleic Acids and a Microarray Comprising Pathogen-Specific Probes

Labeled nucleic acids from the sample are prepared in a solution of 4×SSC buffer, 0.7 μg/μl tRNA, and 0.3% SDS to a total volume of 14.75 μl. The hybridization mixture is denatured at 98° C. for 2 minutes, cooled to 65° C., applied to the microarray, and covered with a 22-mm² cover slip. The slide is placed in a waterproof hybridization chamber for hybridization in a 65° C. water bath for 3 hours. Following hybridization, slides are washed in 1×SSC buffer with 0.06% SDS followed by 2 minutes in 0.06×SSC buffer.

### Example 9

Detection of Aflatoxin Biosynthetic Genes

Aflatoxin biosynthetic genes ver1 (GenBank Accession No. M91369) and nor (GenBank Accession No. U24698) from Aspergillus flavus are amplified using vectors comprising cDNA and/or vector-specific primers. Amplified ver1 and nor products are spotted onto a microarray as described in Example 7. Randomly amplified nucleic acids are prepared from a biological sample, for example as described in Example 2. The randomly amplified nucleic acids are labeled as described in Example 6. Randomly amplified and labeled nucleic acids are hybridized with the microarray as described in Example 8, and duplexes comprising ver1 or nor sequences indicating the presence of aflatoxin biosynthetic genes are detected using a SCANARRAY® 4000 scanning laser confocal fluorescence microscope, which is available from General Scanning, Inc. of Watertown, Mass., United States of America.

### Example 10

Determination of a Detectable Amount of a Pathogen-Specific Probe

Variable amounts of pure pathogen-specific genomic DNA (e.g., 50 ng, 1 ng, and 500 pg, approximately) are each fluorescently labeled as described in Example 6. A. flavus probes are prepared as described in Example 9, and a microarray is prepared, which comprises each of variable amounts (e.g., spotting a same volume of probe preparations having concentrations of 200 ng/ul and 400 ng/ul) of each A. flavus probe. The microarray is prepared using a GMS 417™ arrayer from Affymetrix of Santa Clara, Calif., United States of America. Each sample comprising labeled A. flavus genomic DNA is hybridized with a microarray so prepared using hybridization methods as described in Example 8. Hybridization duplexes are detected using a SCANARRAY® 4000 scanning laser confocal fluorescence microscope, which is available from General Scanning, Inc. of Watertown, Mass., United States of America. A minimal amount of detectable A. flavus ver1 and nor probes is determined as the minimal probe amount generating a detectable fluorescent signal following hybridization.

A detectable amount of pathogen-specific probe can vary according to a variety of factors, including the choice of detectable label, detection methods, probe length, and hybridization conditions. A detectable amount of probe for a particular application can be determined using analogous methods.

### Example 11

Determination of an Amount of Target Nucleic Acids

An amount of nucleic acid derived from a biological sample that can be used to detect a pathogen in the biological sample according to the methods of the present invention can be determined as described herein. Variable amounts of nucleic acids of the biological sample are randomly amplified and labeled, and then hybridized with at least an amount of minimally detectable pathogen-specific probe. A minimal amount of isolated genomic DNA that is suitable for detection of a pathogen according to the methods of the present invention is determined as an amount generating detectable hybridization duplexes. For subsequent hybridization assays employing genomic DNA prepared from a biological sample, at least a minimal amount of genomic DNA so-determined is used.

For example, minimally detectable amounts of A. flavus ver1 and nor probes can be determined as described in Example 10. To determine an amount of target nucleic acid sufficient for detection of A. flavus in a biological sample, isolated genomic DNA is prepared from a biological sample comprising A. flavus genomic DNA, and variable amounts of the isolated genomic DNA are randomly amplified using random primers as described in Example 2. Each randomly amplified sample is labeled and hybridized with a microarray comprising a detectable amount of A. flavus ver1 and nor probes. A minimal amount of isolated genomic DNA that is suitable for detection of A. flavus is determined as an amount generating detectable hybridization duplexes.

A detectable amount of target nucleic acids can vary according to a variety of factors, including but not limited to: infection in a particular sample, the choice of a detectable label, detection methods, probe length, nucleic acid isolation and amplification technique, and hybridization conditions. A minimal amount of target nucleic acids comprising cDNA, organelle DNA, genomic RNA, mRNA, rRNA, or tRNA can be determined using analogous methods.

### Example 12

Veterinary Infectious Disease Test

This Example discloses a veterinary infectious disease test using Southern blot hybridization. The Southern blot tests biological samples for the presence of 2 infectious pathogens: feline parvovirus and Toxoplasma gondii. In addition the blot has a positive control for feline (or canine) genomic DNA. Five (5) pathogen specific PCR products were amplified from the genomic DNA from Toxoplasma gondii and four (4) from feline parvovirus. Two feline specific genes, histone and ferritin, were amplified by PCR from feline genomic DNA. Eleven PCR products total (5 Toxoplasma, 4 parvovirus, 1 histone, 1 ferritin) were run by electrophoresis on an agarose gel and transferred to nitrocellulose paper by Southern blot to provide for immobilization of the pathogen-specific probes on a solid substrate.

Genomic DNA was extracted from the frozen brain of a cat that was confirmed to have been infected with the feline parvovirus by employing PCR. Two micrograms of genomic DNA from the infected cat brain were randomly labeled with radioisotope and hybridized with the test blot. No labeling of the blot was observed. To determine whether or not randomly amplifying this genomic DNA would improve the sensitivity of this assay, degenerate oligo primed (DOP) PCR was employed to first amplify 50 nanograms of genomic DNA, and this amplified product was then radiolabeled. Following hybridization of this radiolabeled amplified probe from infected brain, positive hybridization was observed in two of the four lanes for feline parvovirus as well as in the positive control lanes for ferritin and histone. No labeling was seen in the 5 lanes for Toxoplasma gondii.

This Example demonstrates that random amplification of genomic DNA increases the sensitivity for Southern blot hybridization and indicates its utility in microarray applications. Unamplified DNA from the panleukopenia-infected cat produced no hybridization on the Southern blot array. Randomly amplified genomic DNA from the same infected cat produced strong labeling of the feline specific genomic DNA fragments and hybridization for the panleukopenia-specific DNA.

### Example 13

Amplification of DNA from Infected Corn Sample

The following genomic DNA was blotted onto HYBOND® N+ nylon membranes (Amersham Pharmacia Biotech of Piscataway, N.J., United States of America):

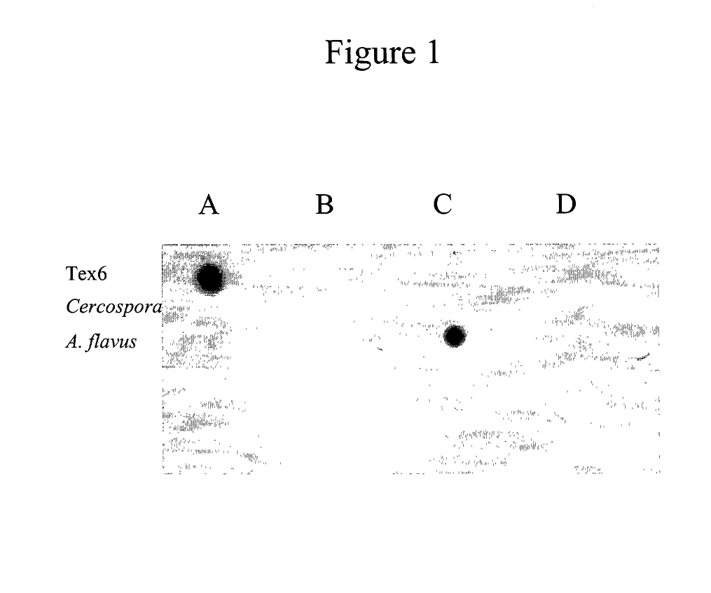

FIG. 1, Row 1—1.5 ug Tex 6 (DNA from corn kernels-variety Tex 6)

FIG. 1, Row 2—1.8 ug Cercospora DNA

FIG. 1, Row 3—1.8 ug A. flavus DNA

Four separate filters were made (FIG. 1; A, B, C, and D).

For the infection of corn kernels, 10 kernels were autoclaved then inoculated with 1×10⁶ spores of A. flavus and allowed to grow for two days at 28° C. Genomic DNA was then isolated. Filters A-D were hybridized with the following labeled samples:

A) 10 μl of 1:10 dilution of A. flavus infected Tex6 DNA (700 ng DNA)

B) 10 μl of 1:1000 dilution of A. flavus infected Tex6 DNA (7 ng DNA)

C) 10 μl of a 50 ul DOP reaction containing 10 μl of 1:1000 dilution of A. flavus infected Tex6 DNA

D) 10 μl of a water control DOP reaction.

DNA was labeled with ³²P using the PRIME-IT II® random prime labeling kit from Stratagene of La Jolla, Calif., United States of America.

FIG. 1, column A shows that 700 ng of ³²P-labeled A. flavus infected Tex6 genomic DNA hybridizes to both Tex6 and A. flavus genomic DNA, though A. flavus signal is less intense. At 7 ng infected DNA (FIG. 1, column B) no hybridization is seen. If 7 ng infected DNA is DOP amplified, both Tex6 (though light) and A. flavus hybridizes (FIG. 1, column C). A water control DOP amplification shows no hybridization (FIG. 1, column D). The amplification was performed using the DOP primer and a modified version of the PCR protocols found in the article by Larsen et al., Cytometry 44:317-325 (2001).

### Example 14

Amplification of DNA from Infected Corn Sample

Ten kernels of Tex 6 corn were autoclaved and inoculated with approximately 1,000,000 spores from either A. flavus or F. verticilliodes. After two days at 28° C., kernels were harvested, frozen in liquid nitrogen and stored at −80° C. DNA was isolated from infected kernels and amplified and labeled using three (3) rounds of ³²P-random primed labeling (PRIME-IT II® kit from Stratagene). The labeled products were purified with a CENTRI SPIN™ 20 column (available from emp Biotech GmbH, Robert-Rössle-Str. 10 D-13125, Berlin, Germany) and allowed to hybridize to the filter.

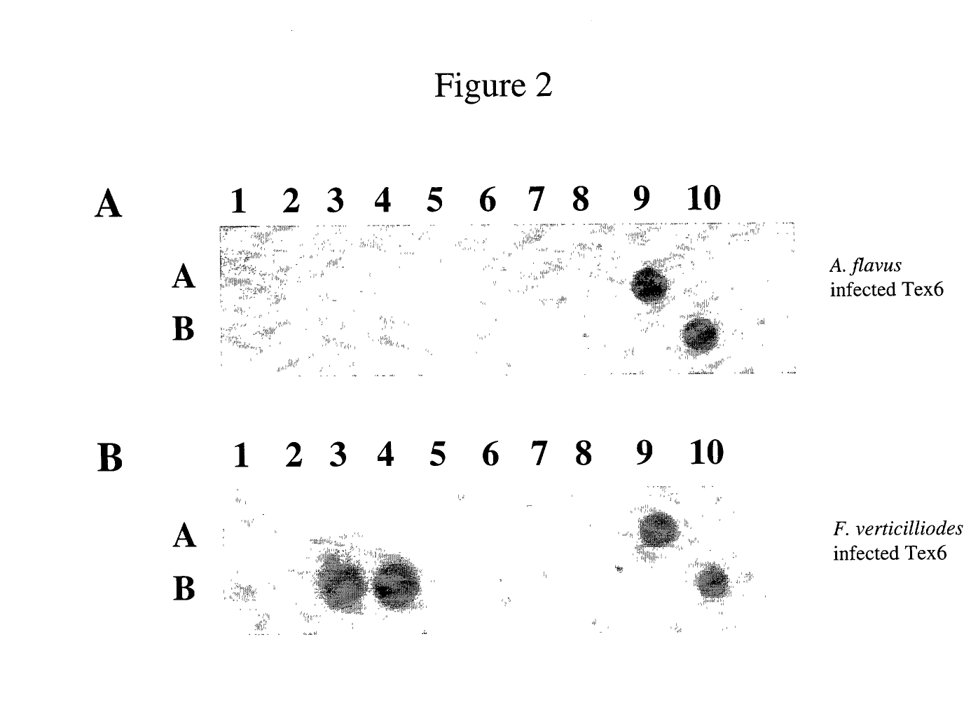

Duplicate filters were produced by blotting 10 μg genomic DNA in duplicate onto HYBOND® C SUPER nitrocellulose (Amersham). The order of blotting was as follows (see FIGS. 2A and 2B):

A1, B1 A. flavus

B3, B4 F. verticilliodes

B6, A7 C. nicotianea

A9, B10 Tex6 corn

As shown in FIG. 2A, one filter was hybridized with DNA isolated from A. flavus infected corn kernels. Spots reflecting hybridization were seen in lanes A1, B1, (reflecting hybridization to A. flavus genomic DNA) and A9, B10 (reflecting hybridization to Tex6 corn genomic DNA) in FIG. 2A. As shown in FIG. 2B, one filter was hybridized with DNA isolated from F. verticilliodes infected corn kernels. Spots reflecting hybridization were seen in B3, B4 (reflecting hybridization to F. verticilliodes genomic DNA) and A9, B10 (reflecting hybridization to Tex6 corn genomic DNA) in FIG. 2B.

### REFERENCES

The references listed below as well as all references cited in the specification are incorporated herein by reference to the extent that they supplement, explain, provide a background for, or teach methodology, techniques and/or compositions employed herein.

Albert et al. (1992) J Virol 66:5627-5630.

Alexay et al. (1996) The International Society of Optical Engineering 2705/63.

Altschul et al. (1990) J Mol Biol 215: 403-410.

Ausubel et al. (1992) Current Protocols in Molecular Biology, John Wiley and Sons, Inc., New York, N.Y.

Barbaux S., et al., J Mol Med (2001) Jun; 79(5-6):329-32

Barrett et al. (1997) in Lee et al., eds., Nucleic Acid Amplification Technologies: Application to Disease Diagnosis, pp. 171-182, Birkhäuser, Boston, Mass., United States of America.

Behr et al. (1999) Science 284:1479-1480.

Bej et al. (1991) Appl Environ Microbiol 57:3529-3534.

Bobrow et al. (1989) J Immunol Methods 125:279-285.

Boom et al. (1990) J Clin Microbiol 28:495-503.

Brush (2001) The Scientist 15(9):25-28.

Buffone et al. (1991) Clin Chem 37:1945-1949.

Busch et al. (1992) Transfusion 32:420-425.

Cano et al. (1993) J Appl Bacteriol 75:247-253.

Cha & Thilly (1993) PCR Methods Appl 3:S18-S29.

Chiodi et al. (1992) J Clin Microbiol 30:255-258.

Clark, ed. (1997) Plant Molecular Biology: A Laboratory Manual, Springer, New York, N.Y.

Csuros (1994) Environmental Sampling and Analysis for Technicians, Lewis Publishers, Boca Raton, Fla.

Dashek, ed. (1997) Methods in Plant Biochemistry and Molecular Biology, CRC Press, Boca Raton, Fla.

Davis & Lau (1997) Hepatology 26:122S-127S.

DeRisi et al. (1996) Nat Genet 14:457-460.

De Saizeu et al. (1998) Nature Biotechnol 16:45-48.

Dubiley et al. (1997) Nuc Acids Res 25:2259-2265.

Dumler & Valsmakis (1999) Am J Clin Pathol 112(1Suppl1):S33-39.

Eberwine et al. (1992) Proc Natl Acad Sci USA 89:3010-3014.

Elfath et al. (2000) Clin Microbiol Rev 13(4):559-570.

Englert (2000) in Schena, ed., Microarray Biochip Technology, pp. 231-246, Eaton Publishing, Natick, Mass., United States of America.

Feng (1992) J Food Protection 55:927-934.

Fisher & Cook (1998) Fundamentals of Diagnostic Mycology, W.B. Saunders Company, Philadelphia, Pa.

Fodor et al. (1991) Science 251:767-773.

Fodor et al. (1993) Nature 364:555-556.

Foo et al. (1992) Biotechniques 12(6):811-814.

Forns & Bukh (1998) Viral Hepatitis 4:1-19.

Fraser et al. (2000) Nature 406(17):799-803.

Gasser (1999) Vet Parasitol 84(3-4):229-258.

Giesendorf et al. (1992) Appl Environ Microbiol 58:3804-3808.

Gingeras et al. (1998) Genome Res 8:435-448.

Glover, ed (1985) DNA Cloning: A Practical Approach, MRL Press, Ltd., Oxford, United Kingdom.

Goldenberger et al. (1997) J Clin Microbiol 35:2733-2739.

Gröndahl et al. (1999) J Clin Microbiol 37:1-7.

Guedon et al. (2000) Anal Chem 72(24):6003-6009.

Guidet (1994) Nuc Acids Res 22(9): 1772.

Günthard et al. (1998) AIDS Res Hum Retroviruses 14:869-876.

Hamel et al. (1995) J Clin Microbiol 33:287-291.

Haymerle et al. (1986) Nuc Acids Res 14:8615-8624.

Heaton et al. (2001) Proc Natl Acad Sci USA 98(7):3701-3704.

Heller et al. (1999) in Schena, ed., DNA Microarrays: A Practical Approach, pp.167-186, Oxford University Press Inc., New York, N.Y.

Henikoff & Henikoff (1989) Proc Natl Acad Sci USA 89:10915.

Hermanson (1990) Bioconjugate Techniques, Academic Press, San Diego, Calif.

Herrewegh et al. (1995) J Clin Microbiol 33:684-689.

Hopkins & Hilton (2001) BioTechniques 30(6):1262-1267.

Izraeli et al. (1991) Nuc Acids Res 19:6051.

Karlin & Altschul (1993) Proc Natl Acad Sci USA 90:5873-5887.

Keasler & Hill (1992) J Food Protection 55:382-384.

Kinzler & Vogelstein (1989) Nuc Acids Res 17(10):3645-3653.

Kittler R., et al., Anal Biochem (2002) January 15; 300(2):237-44

Klausegger et al. (1999) J Clin Microbiol 37:464-466.

Kohsaka & Carson (1994) J Clin Lab Anal 8:425-455.

Kronstad, ed. (2000) Fungal Pathology, Kluwer Academic Publishers, Norwell, Mass., United States of America.

Kwoh et al. (1989) Proc Natl Acad Sci USA 86:1173-1177.

Lanciotti et al. (1992) J Clin Microbiol 30:545-551.

Larsen et al., Cytometry 44:317-325 (2001).

Lawlor et al. (1991) Nature 349:785-788.

Lederberg (1997) Emerg Infect Disease 4:417-423.

Ley et al. (1998) Eur J Clin Microbial Infect Dis 17:247-253.

Liang & Pardee (1993) Nuc Acids Res 21:3269-3275.

Linz et al. (1990) J Clin Chem Clin Biochem 28:5-13.

Lisle et al. (2001) BioTechniques 30(6):1268-1272.

Liu et al. (2000) J Med Microbiol 49(6):493-497.

Liu & Hlady (1996) Coll Sur B 8:25-37.

Louie et al. (2000) CMAJ 163(3):301-309.

Mace et al. (2000) in Shena, ed., Microarray Biochip Technology, pp. 39-64, Eaton Publishing, Natick, Mass., United States of America.

Maier et al. (1994) J Biotechnol 35:191-203.

Maitra & Thakur (1994) Indian J Biochem Biophys 31:97-99.

Maitra & Thakur (1992) Curr Sci 62:586-588.

Maliga et al., eds. (1995) Methods in Plant Molecular Biology: A Laboratory Course Manual, Cold Spring Harbor Laboratory Press, Plainview, N.Y.

Maniatis et al. (1989) Molecular Cloning: A Laboratory Manual, Cold Spring Harbor Press, Cold Spring Harbor, N.Y.

McCaustland et al. (1991) J Virol Methods 35:331-342.

McPherson et al. (1995) PCR 2: A Practical Approach, IRL Press, New York, N.Y.

Millar et al. (1995) Anal Biochem 226:325-330.

Morris & Potter (1997) Emerg Infect Dis 1:435-441.

Morse (1995) Emerg Infect Dis 1:7-15.

Mueller & Wold (1989) Science 246:780-785.

Natarajan et al. (1994) PCR Methods Appl 3:346-350.

Navidid et al. (1992) Am J Hum Genet 50:347-349.

Needleman & Wunsch (1970) J Mol Biol 48:443.

Nelson et al. (2001) Anal Chem 73(1):1-7.

Niblett et al. (2000) Virus Res 71(1-2):97-106.

O'Donnell et al. (1997) Anal Chem 69:2438-2443.

Paladichuk (1999) The Scientist 13(16):20-23.

Pearson & Lipman (1988) Proc Natl Acad Sci USA 85:2444-2448.

Peng et al. (1994) J Clin Pathol 47:605-608.

Persing et al., eds. (1993) Diagnostic Molecular Microbiology—Principles and Applications, American Society for Microbiology, Washington, D.C.

Piétu et al. (1996) Genome Res 6:492-503.

Podzorski et al. (1995) in Murray et al., eds., Manual of Clinical Microbiology, p.130, American Society for Microbiology, Washington, D.C.

Quinn (1997) in Lee et al., eds., Nucleic Acid Ampilification Technologies: Application to Disease Diagnostics, pp. 49-60, Birkhäuser Boston, Cambridge, Mass., United States of America.

Randolph & Waggoner (1995) Nuc Acids Res 25:2923-2929.

Ratner & Castner (1997) in Vickerman, ed., Surface Analysis: The Principal Techniques, John Wiley & Sons, New York.

Ray (1997) J Food Protection 42:346-355.

Reyes & Kim (1991) Mol Cell Probes 5:473-481.

Richardson & Warnock (1993) Fungal Infection: Diagnosis and Management, Blackwell Scientific Publications Inc. of Boston, Mass., United States of America.

Richmond et al. (1999) Nuc Acids Res 27:3821-3835.

Robertson & Walsh-Weller (1998) Methods Mol Biol 98:121-154.

Roos et al. (1995) Parasitol Today 11:148-150.

Rose (2000) in Shena, ed., Microarray Biochip Technology, pp. 19-38, Eaton Publishing, Natick, Mass., United States of America.

Roux (1995) PCR Methods Appl 4:S185-S194.

Rupp et al. (1988) BioTechniques 6:56-60.

Sakallah (2000) Biotechnol Annu Rev 6:141-161.

Salamon et al. (2000) Genome Res 10(12):2044-2054.

Sambrook et al. (1989) Molecular Cloning: A Laboratory Manual, 2^{nd} Ed., Cold Spring Harbor Laboratory Press, Cold Spring Harbor, N.Y.

Sapolsky & Lipshutz (1996) Genomics 33:445-456.

Schena et al. (1995) Science 270:467-470.

Schena et al. (1996) Proc Natl Acad Sci USA 93:10614-10619.

Seth et al. (1986) Gene 42:49-57.

Shalon et al. (1996) Genome Res 6:639-645.

Shoemaker et al. (1996) Nat Genet 14:450-456.

Shriver-Lake (1998) in Cass & Ligler, eds., Immobilized Biomolecules in Analysis, pp.1-14, Oxford Press, Oxford, United Kingdom.

Silhavy et al. (1984) Experiments with Gene Fusions, Cold Spring Harbor Laboratory Press, Cold Spring Harbor, N.Y.

Smith & Waterman (1981) Adv Appl Math 2:482.

Smith et al. (1998) Clin Chem 44(9):2054-2056.

Smith (1998) The Scientist 12(14):21-24.

Snijders et al. (2000) J Clin Pathol 53:289-294.

Southern (1975) J Mol Biol 98:503-517.

Southern et al. (1999) Nat Genet Suppl 21:5-9.

Specter et al., eds. (1998) Rapid Detection of Infectious Agents, Plenum Press, New York, N.Y.

Steel et al. (2000) in Schena, ed., Microarray Biochip Technology, pp. 87-118, Eaton Publishing, Natick, Mass., United States of America.

Storch (2000) Essentials of Diagnostic Virology, Churchill Livingstone, New York, N.Y.

Strain & Chmielewski (2001) BioTechniques 30(6):1286-1291.

Swaminathan & Feng (1994) Annu Rev Microbiol 48:401-426.

Tam et al. (1989) FASEB J 3:1626.

Tanaka et al. (1994) J Gen Virol 75:2691-2698.

Tao et al. (1999) J Bacteriol 181:6425-6440.

Taylor et al. (2001) Biotechniques 30(3):661-669.

Telenius et al. (1992) Genomics 13:718-725.

Theriault et al. (1999) in Schena, ed., DNA Microarrays: A Practical Approach, pp.101-120, Oxford University Press Inc., New York, N.Y.

Tijssen (1993) Laboratory Techniques in Biochemistry and Molecular Biology-Hybridization with Nucleic Acid Probes, part I chapter 2, Elsevier, New York, N.Y.

Tsai & Olson (1992) Appl Environ Microbiol 59:353-357.

U.S. Pat. No. 4,729,947

U.S. Pat. No. 5,196,306

U.S. Pat. No. 5,207,880

U.S. Pat. No. 5,230,781

U.S. Pat. No. 5,346,603

U.S. Pat. No. 5,360,523

U.S. Pat. No. 5,445,934

U.S. Pat. No. 5,534,125

U.S. Pat. No. 5,571,388

U.S. Pat. No. 5,583,001

U.S. Pat. No. 5,731,158

U.S. Pat. No. 5,743,960

U.S. Pat. No. 5,759,778

U.S. Pat. No. 5,843,767

U.S. Pat. No. 5,846,717

U.S. Pat. No. 5,871,918

U.S. Pat. No. 5,916,524

U.S. Pat. No. 5,965,352

U.S. Pat. No. 5,968,745

U.S. Pat. No. 5,985,557

U.S. Pat. No. 5,994,069

U.S. Pat. No. 6,001,567

U.S. Pat. No. 6,086,737

U.S. Pat. No. 6,090,543

U.S. Pat. No. 6,017,696

U.S. Pat. No. 6,027,893

U.S. Pat. No. 6,123,819

U.S. Pat. No. 6,127,127

U.S. Pat. No. 6,162,603

U.S. Pat. No. 6,165,721

U.S. Pat. No. 6,194,149

U.S. Pat. No. 6,197,514

U.S. Pat. No. 6,214,545

U.S. Pat. No. 6,225,059

U.S. Pat. No. 6,245,508

Van Gelder et al. (1990) Proc Natl Acad Sci USA 87:1663-1667.

Vankerckhoven et al. (1994) J Clin Microbiol 30:750-753.

Vignali (2000) J Immunol Methods 243(1-2):243-255.

Wang et al. (1993) Nuc Acids Res 21(17):4153.

Wang et al. (1998) Proc Nat/Acad Sci USA 86:9717-9721.

Warrington et al. (2000) in Shena, ed., Microarray Biochip Technology, pp. 119-148, Eaton Publishing, Natick, Mass., United States of America.

Way et al. (1993) Appl Environ Microbiol 59:1473-1479.

White & Fenner (1994) Medical Virology, 4^{th} Edition, Academic Press, San Diego, Calif.

Williams (1989) BioTechniques 7:762-769.

Williams et al. (1990) Nuc Acids Res 18(22):6531-6535.

Winzeler et al. (1998) Science 281:1194.

WO 01/06013

WO 01/13120

WO 01/14589

WO 01/23082

WO 93/03186

WO 97/14028

WO 99/19515

WO 99/63385

Wodicka et al. (1997) Nat Biotechnol 15:1359-1366.

Worley et al. (2000) in Shena, ed., Microarray Biochip Technology, pp. 65-86, Eaton Publishing, Natick, Mass., United States of America.

Yang et al. (1998) Science 282:2244-2246.

Ye et al. (2001) Hum Mutat 17(4):305-316.

Yershov et al. (1996) Proc Natl Acad Sci USA 93:4319-4918.

Zhang et al. (1992) Proc Natl Acad Sci USA 89:5847-5851.

It will be understood that various details of the invention can be changed without departing from the scope of the invention. Furthermore, the foregoing description is for the purpose of illustration only, and not for the purpose of limitation—the invention being defined by the claims appended hereto.

What is claimed:

1. A method for detecting a pathogen in a biological sample, the method comprising:
(a) procuring a biological sample, wherein the biological sample comprises nucleic acid material;
(b) amplifying the nucleic acid material using random primers to produce a set of random amplicons;
(c) providing one or more pathogen-specific probes;
(d) hybridizing the set of random amplicons with the one or more pathogen-specific probes; and
(e) determining selective hybridization between a random amplicon and a pathogen-specific probe, whereby the presence of a pathogen in a subject is detected.

2. The method of claim 1, wherein the biological sample comprises a clinical sample, a plant sample, an environmental sample, or a food sample.

3. The method of claim 2, wherein the clinical sample is derived from a warm-blooded vertebrate.

4. The method of claim 3, wherein the warm-blooded vertebrate is a human.

5. The method of claim 2, wherein the clinical sample comprises blood; plasma; urine; stool; sputum; a biopsy; a lesional or ulcerous swab; pus; a throat, nose, or nasopharyngeal swab; mucus; an endotracheal aspirate; bronchoalveolar lavage; a conjuctival or corneal swab; bone marrow; cerebrospinal fluid; skin; hair; nails; a cell culture; or combinations thereof.

6. The method of claim 2, wherein the plant sample comprises a seed, a leaf, a stem, a root, a flower, a fruit, a plant culture, or combinations thereof.

7. The method of claim 2, wherein the environmental sample is a water sample or a soil sample.

8. The method of claim 1, wherein the nucleic acid material is deoxyribonucleic acid material, ribonucleic acid material, or a combination thereof.

9. The method of claim 1, wherein the set of random amplicons further comprises a detectable label.

10. The method of claim 9, wherein the detectable label is a fluorophore or an epitope.

11. The method of claim 9, wherein the detectable label is incorporated during synthesis of the random amplicons or following synthesis of the random amplicons.

12. The method of claim 1, wherein the one or more pathogen-specific probes each comprises a nucleotide sequence derived from a bacterium, a fungus, a virus, a protozoan, or a parasite.

13. The method of claim 12, wherein the one or more pathogen-specific probes each comprises a nucleotide sequence of a pathogen gene.

14. The method of claim 13, wherein the one or more pathogen-specific probes each comprises a nucleotide sequence encoding a pathogen polypeptide.

15. The method of claim 1, wherein at least one of the one or more pathogen-specific probes further comprises a detectable label.

16. The method of claim 15, wherein the detectable label is a fluorophore or an epitope.

17. The method of claim 15, further comprising a number, n, of different pathogen-specific probes, and a number, n, of different detectable labels, each pathogen-specific probe comprising a different detectable label.

18. The method of claim 17, wherein each of the different detectable labels comprises a fluorophore or an epitope.

19. The method of claim 1, wherein the one or more pathogen-specific probes are immobilized on a solid substrate comprising a plurality of identifying positions, each of the one or more pathogen-specific probes occupying one of the plurality of identifying positions.

20. The method of claim 19, wherein the solid substrate comprises silicon, glass, plastic, polyacrylamide, a polymer matrix, an agarose gel, a polyacrylamide gel, an organic membrane, or an inorganic membrane.

21. A method for detecting a pathogen in a biological sample, the method comprising:
(a) procuring a biological sample, wherein the biological sample comprises nucleic acid material;
(b) amplifying the nucleic acid material using random primers to produce a set of random amplicons;
(c) providing one or more pathogen-specific probe sets;
(d) hybridizing the set of random amplicons with the one or more pathogen-specific probe sets; and
(e) determining selective hybridization between a random amplicon and a pathogen-specific probe set, whereby the presence of a pathogen in a biological sample is detected.

22. The method of claim 21, wherein the biological sample comprises a clinical sample, a plant sample, an environmental sample, or a food sample.

23. The method of claim 22, wherein the clinical sample is derived from a warm-blooded vertebrate.

24. The method of claim 23, wherein the warm-blooded vertebrate is a human.

25. The method of claim 22, wherein the clinical sample comprises blood; plasma; urine; stool; sputum; a biopsy; a lesional or ulcerous swab; pus; a throat, nose, or nasopharyngeal swab; mucus; an endotracheal aspirate; bronchoalveolar lavage; a conjuctival or corneal swab; bone marrow; cerebrospinal fluid; skin; hair; nails; a cell culture; or combinations thereof.

26. The method of claim 22, wherein the plant sample comprises a seed, a leaf, a stem, a root, a flower, a fruit, or combinations thereof.

27. The method of claim 22, wherein the environmental sample is a water sample or a soil sample.

28. The method of claim 21, wherein the nucleic acid material is deoxyribonucleic acid material, ribonucleic acid material, or a combination thereof.

29. The method of claim 21, wherein the set of random amplicons further comprises a detectable label.

30. The method of claim 29, wherein the detectable label is a fluorophore or an epitope.

31. The method of claim 29, wherein the detectable label is incorporated during synthesis of the random amplicons or following synthesis of the random amplicons.

32. The method of claim 21, wherein each of the one or more pathogen-specific probe sets comprises two or more pathogen-specific probes, each of the two or more pathogen-specific probes of a pathogen-specific probe set comprising a different nucleotide sequence of a same pathogen.

33. The method of claim 32, wherein each of the one or more pathogen-specific probe sets comprises at least about four or five pathogen-specific probes, each of the at least about four or five pathogen-specific probes comprising a different nucleotide sequence of a same pathogen.

34. The method of claim 33, wherein each of the one or more pathogen-specific probe sets comprises about 6 to 10 pathogen-specific probes, each of the about 6 to 10 pathogen-specific probes comprising a different nucleotide sequence of a same pathogen.

35. The method of claim 32, wherein each of one or more pathogen-specific probes of the one or more pathogen-specific probe sets comprises a nucleotide sequence derived from a bacterium, a fungus, a virus, a protozoan, or a parasite.

36. The method of claim 35, wherein each of the one or more pathogen-specific probes of the one or more pathogen-specific probe sets comprises a nucleotide sequence of a pathogen gene.

37. The method of claim 36, wherein each of the one or more pathogen-specific probes of the one or more pathogen-specific probe sets comprises a nucleotide sequence encoding a pathogen polypeptide.

38. The method of claim 21, wherein the one or more pathogen-specific probe sets further comprises a detectable label.

39. The method of claim 38, wherein the detectable label is a fluorophore or an epitope.

40. The method of claim 39, further comprising a number, n, of different pathogen-specific probe sets, and a number, n, of different detectable labels, each pathogen-specific probe set comprising a different detectable label.

41. The method of claim 40, wherein each of the different detectable labels comprises a fluorophore or an epitope.

42. The method of claim 21, wherein the one or more pathogen-specific probe sets are immobilized on a solid substrate comprising a plurality of identifying positions, each of the one or more pathogen-specific probe sets occupying one of the plurality of identifying positions.

43. The method of claim 42, wherein the solid substrate comprises silicon, glass, plastic, polyacrylamide, a polymer matrix, an agarose gel, a polyacrylamide gel, an organic membrane, or an inorganic membrane.

44. A microarray for detecting a pathogen in a biological sample comprising:
(a) a solid support comprising a plurality of identifying positions; and
(b) one or more pathogen-specific probe sets, each probe set occupying one of the plurality of identifying positions on the solid support.

45. The microarray of claim 44, wherein the solid substrate comprises silicon, glass, plastic, polyacrylamide, a polymer matrix, an agarose gel, a polyacrylamide gel, an organic membrane, or an inorganic membrane.

46. The microarray of claim 44, wherein each of the one or more pathogen-specific probe sets comprises two or more pathogen-specific probes, each of the two or more pathogen-specific probes of a pathogen-specific probe set comprising a different nucleotide sequence of a same pathogen.

47. The microarray of claim 44, wherein each of the one or more pathogen-specific probe sets comprises at least about three, four or five pathogen-specific probes, each of the about three, four or five pathogen-specific probes comprising a different nucleotide sequence of a same pathogen.

48. The method of claim 44, wherein each of the one or more pathogen-specific probe sets comprises about 6 to 10 pathogen-specific probes, each of the about 6 to 10 pathogen-specific probes comprising a different nucleotide sequence of a same pathogen.

49. The microarray of claim 44, wherein each of one or more pathogen-specific probes of the one or more pathogen-specific probe sets comprises a nucleotide sequence derived from a bacterium, a fungus, a virus, a protozoan, or a parasite.

50. The microarray of claim 49, wherein each of the one or more pathogen-specific probes of the one or more pathogen-specific probe sets comprises a nucleotide sequence of a pathogen gene.

51. The microarray of claim 50, wherein each of the one or more pathogen-specific probes of the one or more pathogen-specific probe sets comprises a nucleotide sequence encoding a pathogen polypeptide.


SEQUENCE LISTING (1 sequence)

SEQ ID NO: 1 - DNA, 57 bp, Bacteriophage T7
aaacgacggccagtgaattgtaatacgactcactataggcgcttttttttttttttt